(19) United States
(12) Patent Application Publication
(10) Pub. No.: US 2004/0229781 A1
(43) Pub. Date: Nov. 18, 2004

(54) Compounds and methods for treating and preventing exercise-induced cardiac arrhythmias

(76) Inventors: Andrew Robert Marks, Larchmont, NY (US); Donald W. Landry, New York, NY (US); Shi Xian Deng, White Plains, NY (US); Zhen Zhuang Cheng, Elmhurst, NY (US)

Correspondence Address: Leslie Gladstone Restaino, 163 Madison Avenue, P.O. Box 1989, Morristown, NJ, 07962-1989, US

(21) Appl. No.: 10/680,988

(22) Filed: Oct. 7, 2003

Related U.S. Application Data
(63) Continuation-in-part of application No. 10/608,723, filed on Jun. 26, 2003.
Application No. 10/608,723 is a continuation-in-part of application No. 10/288,606, filed on Nov. 5, 2002.
Application No. 10/288,606 is a continuation of application No. 09/568,474, filed on May 10, 2000, now Pat. No. 6,489,125.

(51) Int. Cl. A61K 38/17

(52) U.S. Cl. 514/12

(57) ABSTRACT

The present invention provides a method for limiting or preventing a decrease in the level of RyR2-bound FKBP12.6 in a subject, a method for treating or preventing exercise-induced cardiac arrhythmia in a subject, and a method for preventing exercise-induced sudden cardiac death in a subject. Also provided are uses of JTV-519 in these methods. The present invention further provides methods for identifying agents for use in preventing exercise-induced sudden cardiac death, as well as agents identified by such methods. Also provided are methods for preventing exercise-induced sudden cardiac death by administering these agents. Additionally, the present invention provides methods for synthesizing JTV-519, radio-labeled JTV-519, and 1,4-benzothiazepine intermediates and derivatives.

### RELATED APPLICATIONS

This application is a continuation-in-part of U.S. patent application Ser. No. 10/608,723, filed on Jun. 26, 2003, which is a continuation-in-part of U.S. patent application Ser. No. 10/288,606, filed on Nov. 5, 2002, which is a continuation of U.S. patent application Ser. No. 09/568,474, filed on May 10, 2000, now U.S. Pat. No. 6,489,125 B1, issued on Dec. 3, 2002, the contents of which are hereby incorporated by reference herein.

### STATEMENT OF GOVERNMENT INTEREST

This invention was made with government support under NIH Grant No. PO1 HL 67849-01. As such, the United States government has certain rights in this invention.

### BACKGROUND OF THE INVENTION

Heart failure is a leading cause of mortality and morbidity, world wide. In the more severe cases of heart failure (New York Heart Association class IV), the 2-year mortality rate is over 50% (Braunwald, E. B., Heart Disease, 4^{th} ed. (Philadelphia: W. B. Saunders Co., 1992)). Cardiac arrhythmia, a common feature of heart failure, results in many of the deaths associated with the disease. In particular, approximately 50% of all patients with heart disease die from fatal cardiac arrhythmias. Some ventricular arrhythmias in the heart are rapidly fatal—a phenomenon referred to as “sudden cardiac death” (SCD). However, fatal ventricular arrhythmias may also occur in young, otherwise-healthy individuals who are not known to have structural heart disease. In fact, ventricular arrhythmia is the most common cause of sudden death in otherwise-healthy individuals.

Catecholaminergic polymorphic ventricular tachycardia (CPVT) is an inherited disorder in individuals with structurally-normal hearts. It is characterized by stress-induced ventricular tachycardia—a lethal arrhythmia that may cause sudden cardiac death. In subjects with CPVT, physical exertion and/or stress induce bidirectional and/or polymorphic ventricular tachycardias that lead to SCD in the absence of detectable structural heart disease (Laitinen et al., Mutations of the cardiac ryanodine receptor (RyR2) gene in familial polymorphic ventricular tachycardia. Circulation, 103:485-90, 2001; Leenhardt et al., Catecholaminergic polymorphic ventricular tachycardia in children: a 7-year follow-up of 21 patients. Circulation, 91:1512-19, 1995; Priori et al., Clinical and molecular characterization of patients with catecholaminergic polymorphic ventricular tachycardia. Circulation, 106:69-74, 2002; Priori et al., Mutations in the cardiac ryanodine receptor gene (hRyR2) underlie catecholaminergic polymorphic ventricular tachycardia. Circulation, 103:196-200, 2001; Swan et al., Arrhythmic disorder mapped to chromosome 1 q42-q43 causes malignant polymorphic ventricular tachycardia in structurally normal hearts. J. Am. Coll. Cardiol., 34:2035-42, 1999). CPVT is predominantly inherited in an autosomal-dominant fashion. Individuals with CPVT have ventricular arrhythmias when subjected to exercise, but do not develop arrhythmias at rest. Linkage studies and direct sequencing have identified mutations in the human RyR2 gene, on chromosome 1q42-q43, in individuals with CPVT (Laitinen et al., Mutations of the cardiac ryanodine receptor (RyR2) gene in familial polymorphic ventricular tachycardia. Circulation, 103:485-90, 2001; Priori et al., Mutations in the cardiac ryanodine receptor gene (hRyR2) underlie catecholaminergic polymorphic ventricular tachycardia. Circulation, 103:196-200, 2001; Swan et al., Arrhythmic disorder mapped to chromosome 1q42-q43 causes malignant polymorphic ventricular tachycardia in structurally normal hearts. J. Am. Coll. Cardiol., 34:2035-42, 1999).

Heart failure is characterized by a progressive decrease in the contractile function of cardiac muscle, which leads to hypoperfusion of critical organs. The contraction of heart muscle, and other striated muscle, is initiated when calcium (Ca²⁺) is released from the sarcoplasmic reticulum (SR) into the surrounding cytoplasm. Calcium-release channels on the SR, including ryanodine receptors (RyRs), are required for excitation-contraction (EC) coupling (i.e., coupling of an action potential to a muscle cell's contraction). There are three types of ryanodine receptors, all of which are highly-related Ca²⁺ channels: RyR1, RyR2, and RyR3. RyR1 is found in skeletal muscle, RyR2 is found in the heart, and RyR3 is located in the brain. The type 2 ryanodine receptor (RyR2) is the major Ca²⁺-release channel required for EC coupling and muscle contraction in cardiac striated muscle.

RyR2 channels are packed into dense arrays in specialized regions of the SR that release intracellular stores of Ca²⁺, and thereby trigger muscle contraction (Marx et al., Coupled gating between individual skeletal muscle Ca²⁺ release channels (ryanodine receptors). Science, 281:818-21, 1998). During EC coupling, depolarization of the cardiac-muscle cell membrane, in phase zero of the action potential, activates voltage-gated Ca²⁺ channels. In turn, Ca²⁺ influx through these channels initiates Ca²⁺ release from the SR via RyR2, in a process known as Ca²⁺-induced Ca²⁺ release (Fabiato, A., Calcium-induced release of calcium from the cardiac sarcoplasmic reticulum. Am. J. Physiol., 245:C₁-C₁₄, 1983; Nabauer et al., Regulation of calcium release is gated by calcium current, not gating charge, in cardiac myocytes. Science, 244:800-03, 1989). The RyR2-mediated, Ca²⁺-induced Ca²⁺ release then activates the contractile proteins which are responsible for cardiac muscle contraction.

RyR2 is a protein complex comprising four 565,000-dalton RyR2 polypeptides in association with four 12,000-dalton FK506 binding proteins (FKBPs), specifically FKBP12.6 proteins. FKBPs are cis-trans peptidyl-prolyl isomerases that are widely expressed, and serve a variety of cellular functions (Marks, A. R., Cellular functions of immunophilins. Physiol. Rev., 76:631-49, 1996). FKBP12 proteins are tightly bound to, and regulate the function of, the skeletal ryanodine receptor, RyR1 (Brillantes et al., Stabilization of calcium release channel (ryanodine receptor) function by FK506-binding protein. Cell, 77:513-23, 1994; Jayaraman et al., FK506 binding protein associated with the calcium release channel (ryanodine receptor). J. Biol. Chem., 267:9474-77, 1992); the cardiac ryanodine receptor, RyR2 (Kaftan et al., Effects of rapamycin on ryanodine receptor/Ca(2+)-release channels from cardiac muscle. Circ. Res., 78:990-97, 1996); a related intracellular Ca²⁺-release channel, known as the type 1 inositol 1,4,5-triphosphate receptor (IP3R1) (Cameron et al., FKBP12 binds the inositol 1,4,5-trisphosphate receptor at leucine-proline (1400-1401) and anchors calcineurin to this FK506-like domain. J. Biol. Chem., 272:27582-88, 1997); and the type I transforming growth factor β (TGFβ) receptor (TβRI) (Chen et al., Mechanism of TGFbeta receptor inhibition by FKBP12. EMBO J, 16:3866-76, 1997). FKBP12.6 binds to the RyR2 channel (one molecule per RyR2 subunit), stabilizes RyR2-channel function (Brillantes et al., Stabilization of calcium release channel (ryanodine receptor) function by FK506-binding protein. Cell, 77:513-23, 1994), and facilitates coupled gating between neighboring RyR2 channels (Marx et al., Coupled gating between individual skeletal muscle Ca²⁺ release channels (ryanodine receptors). Science, 281:818-21, 1998), thereby preventing aberrant activation of the channel during the resting phase of the cardiac cycle.

Failing hearts (e.g., in patients with heart failure and in animal models of heart failure) are characterized by a maladaptive response that includes chronic hyperadrenergic stimulation (Bristow et al., Decreased catecholamine sensitivity and beta-adrenergic-receptor density in failing human hearts. N. Engl. J. Med., 307:205-11, 1982). The pathogenic significance of this stimulation in heart failure is supported by therapeutic strategies that decrease β-adrenergic stimulation and left ventricular myocardial wall stress, and potently reverse ventricular remodeling (Barbone et al., Comparison of right and left ventricular responses to left ventricular assist device support in patients with severe heart failure: a primary role of mechanical unloading underlying reverse remodeling. Circulation, 104:670-75, 2001; Eichhorn and Bristow, Medical therapy can improve the biological properties of the chronically failing heart. A new era in the treatment of heart failure. Circulation, 94:2285-96, 1996). In heart failure, chronic β-adrenergic stimulation is associated with the activation of β-adrenergic receptors in the heart, which, through coupling with G-proteins, activate adenylyl cyclase and thereby increase intracellular cAMP concentration. cAMP activates cAMP-dependent protein kinase (PKA), which has been shown to induce hyperphosphorylation of RyR2.

The hyperphosphorylation of RyR2 has been proposed as a factor contributing to depressed contractile function and arrhythmogenesis in heart failure (Marks et al., Progression of heart failure: is protein kinase a hyperphosphorylation of the ryanodine receptor a contributing factor? Circulation, 105:272-75, 2002; Marx et al., PKA phosphorylation dissociates FKBP12.6 from the calcium release channel (ryanodine receptor): defective regulation in failing hearts. Cell, 101:365-76, 2000). Consistent with this hypothesis, PKA hyperphosphorylation of RyR2 in failing hearts has been demonstrated in vivo, both in animal models and in patients with heart failure undergoing cardiac transplantation (Antos et al., Dilated cardiomyopathy and sudden death resulting from constitutive activation of protein kinase A. Circ. Res., 89:997-1004, 2001; Marx et al., PKA phosphorylation dissociates FKBP12.6 from the calcium release channel (ryanodine receptor): defective regulation in failing hearts. Cell, 101:365-76, 2000; Ono et al., Altered interaction of FKBP12.6 with ryanodine receptor as a cause of abnormal Ca⁽²⁺⁾ release in heart failure. Cardiovasc. Res., 48:323-31, 2000; Reiken et al., Beta-adrenergic receptor blockers restore cardiac calcium release channel (ryanodine receptor) structure and function in heart failure. Circulation, 104:2843-48, 2001; Semsarian et al., The L-type calcium channel inhibitor diltiazem prevents cardiomyopathy in a mouse model. J. Clin. Invest., 109:1013-20, 2002; Yano et al., Altered stoichiometry of FKBP12.6 versus ryanodine receptor as a cause of abnormal Ca(2+) leak through ryanodine receptor in heart failure. Circulation, 102:2131-36, 2000).

In failing hearts, the hyperphosphorylation of RyR2 by PKA induces the dissociation of the regulatory FKBP12.6 subunit from the RyR2 channel (Marx et al., PKA phosphorylation dissociates FKBP12.6 from the calcium release channel (ryanodine receptor): defective regulation in failing hearts. Cell, 101:365-76, 2000). This causes marked changes in the biophysical properties of the RyR2 channel. Such changes are evidenced by increased open probability (Po), due to an increased sensitivity to Ca²⁺-dependent activation (Brillantes et al., Stabilization of calcium release channel (ryanodine receptor) function by FK506-binding protein. Cell, 77:513-23, 1994; Kaftan et al., Effects of rapamycin on ryanodine receptor/Ca(2+)-release channels from cardiac muscle. Circ. Res., 78:990-97, 1996); destabilization of the channel, resulting in subconductance states; and impaired coupled gating of the channels, resulting in defective EC coupling and cardiac dysfunction (Marx et al., Coupled gating between individual skeletal muscle Ca2+ release channels (ryanodine receptors). Science, 281:818-21, 1998). Thus, PKA-hyperphosphorylated RyR2 is very sensitive to low-level Ca²⁺ stimulation, and this manifests itself as an SR Ca²⁺ leak through the hyperphosphorylated channel.

In structurally-normal hearts, a similar phenomenon may be at work. Specifically, it is known that exercise and stress induce the release of catecholamines that activate β-adrenergic receptors in the heart. Activation of the β-adrenergic receptors leads to hyperphosphorylation of RyR2 channels. Moreover, evidence suggests that the hyperphosphorylation of RyR2 resulting from β-adrenergic-receptor activation renders mutated RyR2 channels more likely to open in the relaxation phase of the cardiac cycle, increasing the likelihood of arrhythmias.

Cardiac arrhythmias are known to be associated with SR Ca²⁺ leaks in structurally-normal hearts. In these cases, the most common mechanism for induction and maintenance of ventricular tachycardia is abnormal automaticity. One form of abnormal automaticity, known as triggered arrhythmia, is associated with aberrant release of SR Ca²⁺, which initiates delayed after-depolarizations (DADs) (Fozzard, H. A., Afterdepolarizations and triggered activity. Basic Res. Cardiol., 87:105-13, 1992; Wit and Rosen, Pathophysiologic mechanisms of cardiac arrhythmias. Am. Heart J., 106:798-811, 1983). DADs, which can trigger fatal ventricular arrhythmias, are abnormal depolarizations in cardiomyocytes that occur after repolarization of a cardiac action potential. The molecular basis for the abnormal SR Ca²⁺ release that results in DADs has not been fully elucidated. DADs are known, however, to be blocked by ryanodine, providing evidence that RyR2 may play a key role in the pathogenesis of this aberrant Ca²⁺ release (Marban et al., Mechanisms of arrhythmogenic delayed and early afterdepolarizations in ferret ventricular muscle. J. Clin. Invest., 78:1185-92, 1986; Song and Belardinelli, ATP promotes development of afterdepolarizations and triggered activity in cardiac myocytes. Am. J. Physiol., 267:H2005-11, 1994).

In view of the foregoing, it is clear that leaks in RyR2 channels are associated with a number of pathological states—in both diseased hearts and structurally-normal hearts. Accordingly, methods to repair the leaks in RyR2 could prevent fatal arrhythmias in millions of patients.

JTV-519 (4-[3-(4-benzylpiperidin-1-yl)propionyl]-7-methoxy-2,3,4,5-tetrahydro-1,4-benzothiazepine monohydrochloride; also known as k1201), a derivative of 1,4-benzothiazepine, is a new modulator of calcium-ion channels. In addition to regulating Ca²⁺ levels in myocardial cells, JTV-519 also modulates the Na⁺ current and the inward-rectifier K⁺ current in guinea pig ventricular cells, and inhibits the delayed-rectifier K⁺ current in guinea pig atrial cells. Studies have shown that JTV-519 has a strong cardioprotective effect against catecholamine-induced myocardial injury, myocardial-injury-induced myofibrillar overcontraction, and ischemia/reperfusion injury. In experimental myofibrillar overcontraction models, JTV-519 demonstrated greater cardioprotective effects than propranolol, verapamil, and diltiazem. Experimental data also suggest that JTV-519 effectively prevents ventricular ischemia/reperfusion by reducing the level of intracellular Ca²⁺ overload in animal models.

### SUMMARY OF THE INVENTION

The present invention is based upon the surprising discovery that RyR2 is a target for preventing cardiac arrhythmias that cause exercise-induced sudden cardiac death (SCD). As described herein, the inventors made mutant RyR2 channels with 7 different CPVT mutations, and studied their functions. All 7 mutants had functional defects that resulted in channels that became leaky (an SR calcium leak) when stimulated during exercise. The inventors' study is the first to identify a mechanism by which the SR calcium leak causes DADs. Remarkably, the defect in the mutant CPVT channels made the channels look like the leaky channels in the hearts of patients with end-stage heart failure—a disorder characterized by a high incidence of fatal cardiac arrhythmias. Therefore, the inventors demonstrate herein that the mechanism for the VT in CPVT is the same as the mechanism for VT in heart failure.

The inventors also disclose herein that the drug JTV-519 (k201), a member of the 1,4 benzothiazepine family of compounds, repairs the leak in RyR2 channels. As the inventors show herein, JTV-519 enhances binding of FKBP12.6 to PKA-phosphorylated RyR2, and to mutant RyR2s that otherwise have reduced affinity for, or do not bind to, FKBP12.6. This action of JTV-519 fixes the leak in RyR2 that triggers fatal cardiac arrhythmias (cardiac death) and contributes to heart muscle dysfunction in heart failure. In addition, the inventors have developed a novel synthesis for JTV-519, as well as a radio-labeled version of the drug.

Accordingly, in one aspect, the present invention provides a method for limiting or preventing a decrease in the level of RyR2-bound FKBP12.6 in a subject who is a candidate for exercise-induced cardiac arrhythmia, by administering to the subject an amount of JTV-519 effective to prevent a decrease in the level of RyR2-bound FKBP12.6 in the subject. Also provided is a use of JTV-519 in a method for limiting or preventing a decrease in the level of RyR2-bound FKBP12.6 in a subject who is a candidate for exercise-induced cardiac arrhythmia.

In another aspect, the present invention provides a method for treating or preventing exercise-induced cardiac arrhythmia in a subject, by administering JTV-519 to the subject in an amount effective to treat or prevent the exercise-induced cardiac arrhythmia in the subject. Also provided is a use of JTV-519 in a method for treating or preventing exercise-induced cardiac arrhythmia in a subject.

In still another aspect, the present invention provides a method for preventing exercise-induced sudden cardiac death in a subject, by administering to the subject JTV-519 in an amount effective to prevent exercise-induced sudden cardiac death in the subject. Also provided is a use of JTV-519 in a method for preventing exercise-induced sudden cardiac death in a subject.

In yet another aspect, the present invention provides a method for identifying an agent for use in preventing exercise-induced sudden cardiac death, by: (a) obtaining or generating a culture of cells containing RyR2; (b) contacting the cells with a candidate agent; (c) exposing the cells to one or more conditions known to increase phosphorylation of RyR2 in cells; and (d) determining if the agent prevents a decrease in the level of RyR2-bound FKBP12.6 in the cells. The method may further comprise the step of: (e) determining if the agent has an effect on an RyR2-associated biological event in the cells. Also provided are an agent identified by the method, and a method for preventing exercise-induced sudden cardiac death in a subject, by administering the agent to the subject in an amount effective to prevent exercise-induced sudden cardiac death in the subject.

In a further aspect, the present invention provides a method for identifying an agent for use in preventing exercise-induced sudden cardiac death, by: (a) obtaining or generating an animal containing RyR2; (b) administering a candidate agent to the animal; (c) exposing the animal to one or more conditions known to increase phosphorylation of RyR2 in cells; and (d) determining if the agent increases binding between FKBP12.6 and RyR2 in the animal. The may further comprise the step of: (e) determining if the agent has an effect on an RyR2-associated biological event in the animal. Also provided are an agent identified by the method, and a method for preventing exercise-induced sudden cardiac death in a subject, by administering the agent to the subject in an amount effective to prevent exercise-induced sudden cardiac death in the subject.

In still another aspect, the present invention provides methods for synthesizing JTV-519 and 1,4-benzothiazepine intermediates and derivatives, including the following:

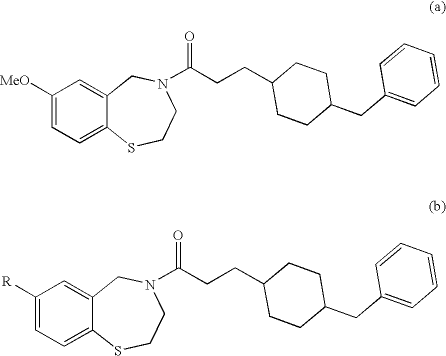

wherein R=OR′, SR′, NR′, alkyl, or halide and R′=alkyl, aryl, or H, and wherein R can be at position 2, 3, 4, or 5;

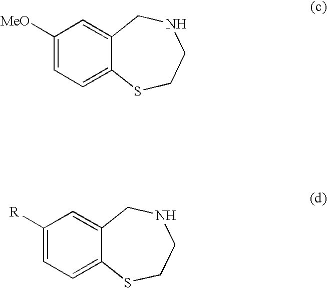

wherein R=OR′, SR′, NR′, alkyl, or halide and R′=alkyl, aryl, or H, and wherein R can be at position 2, 3, 4, or 5;

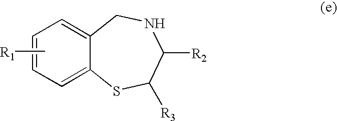

wherein R₁=n-MeO, n-MeS, or n-alkyl, and n=6, 7, 8, or 9; wherein R₂=alkyl; and wherein R₃=alkyl.

In a further aspect, the present invention provides a method for synthesizing radio-labeled JTV-519.

Additional aspects of the present invention will be apparent in view of the description which follows.

### BRIEF DESCRIPTION OF THE FIGURES

FIG. 1 demonstrates that JTV-519 prevents exercise-induced ventricular arrhythmias in FKBP12.6^{+/-} mice. (A) Representative ambulatory electrocardiograms of an untreated FKBP12.6^{+/-} mouse, an FKBP12.6^{+/-} mouse treated with JTV-519, and an FKBP12.6^{-/-} mouse treated with JTV-519. There were no significant differences in heart rate, or in any of the measured ECG parameters. (B) upper tracing: Example of sustained polymorphic ventricular tachycardia, recorded in an untreated FKBP12.6^{+/-} mouse subjected to exercise testing and injection with 1.0 mg/kg epinephrine. middle tracing: Electro-cardiogram of a JTV-519-treated FKBP12.6^{+/-} mouse following the same protocol; no arrhythmias were detected. bottom tracing: Exercise-induced ventricular tachycardia (VT) in an FKBP12.6^{-/-} mouse treated with JTV-519. The dotted line represents 16.31 seconds of VT that are not shown in the figure. ‘P’ indicates a P-wave, which is indicative of sinus rhythm following ventricular tachycardia. (C) Bar graph showing quantification of sudden cardiac death (left), sustained ventricular tachycardias (>10 beats, middle), and non-sustained ventricular tachycardias (3-10 abnormal beats, right) in FKBP12.6^{+/-} and FKBP12.6^{-/-} mice, either treated or not treated with JTV-519, respectively. It should be noted that treatment with JTV-519 completely prevented exercise- and epinephrine-induced arrhythmias in FKBP12.6^{+/-} mice treated with JTV-519 (n=9), as compared with untreated FKBP12.6+/−mice (n=10) or JTV-519-treated FKBP12.6^{-/-} mice (n=5), suggesting that JTV-519 prevents arrhythmias and sudden death in FKBP12.6^{+/-} mice by rebinding FKBP12.6 to RyR2.

FIG. 2 shows that JTV-519 prevents exercise-induced sudden cardiac death (SCD) by increasing the affinity of FKBP12.6 for RyR2 in FKBP12.6^{+/-} mice. (A-B) Cardiac ryanodine receptors (RyR2) were immunoprecipitated using RyR2-5029 antibody. Shown are immunoblots (A) and bar graphs (B) representing the quantified amounts of RyR2, PKA-phosphorylated RyR2 (RyR2-pSer²⁸⁰⁹ antibody), and FKBP12.6 in wild-type (FKBP12.6^{+/+}) mice, FKBP12.6^{+/-} mice, and FKBP12.6^{-/-} under resting conditions, and following exercise, either in the absence or presence of JTV-519, respectively. Under resting conditions, ˜70% of FKBP12.6 is associated with RyR2 in FKBP12.6^{+/-} mice. Following exercise testing, the amount of FKBP12.6 associated with the RyR2 complex was dramatically decreased in FKBP12.6^{+/-} mice, but this could be rescued by treatment with JTV-519. (C) RyR2 single channels were isolated from hearts obtained following exercise testing and epinephrine injection. Shown are channels from FKBP12.6^{+/-} mice, with and without pre-treatment with JTV-519, and channels from FKBP12.6^{-/-} mice following JTV-519 pre-treatment. It should be noted that RyR2-channel function was normalized in the exercised FKBP12.6^{+/-} mouse treated with JTV-519. The representative single channel from an exercised FKBP12.6^{-/-} mouse after JTV-519 treatment shows that FKBP12.6 in the heart is required for the action of JTV-519. The dotted lines represent incomplete channel openings, or ‘subconductance’ openings, and are indicative of FKBP12.6-depleted RyR2 channels. Tracings on the left represent 5.0 sec, while tracings on the right represent 500 msec. In the figure, Po=open probability; To=average open times; Tc=average closed times; and c=closed state of the channel. (D) Summary bar graph showing average open probabilities of single RyR2 channels (see above). JTV-519 dramatically reduces the open probability of RyR2 from FKBP12.6^{+/-} mice following exercise testing at diastolic calcium concentrations (150 nM).

FIG. 3 illustrates JTV-519-normalized RyR2-channel gating by increased FKBP12.6 binding affinity to PKA-phosphorylated RyR2 channels. (A, B) Canine cardiac SR membranes (A) and recombinantly-expressed RyR2 channels (B) were prepared as described previously (Kaftan et al., Effects of rapamycin on ryanodine receptor/Ca⁽²⁺⁾-release channels from cardiac muscle. Circ. Res., 78:990-97, 1996). (A) Ryanodine receptors (RyR2) were phosphorylated with PKA catalytic subunit (40 U; Sigma Chemical Co., St. Louis, Mo.), in the presence or absence of the PKA inhibitor, PKI₅₋₂₄, in phosphorylation buffer (8 mM MgCl₂, 10 mM EGTA, and 50 mM Tris/PIPES; pH 6.8). Samples were centrifuged at 100,000×g for 10 min, and washed three times in imidazole buffer (10 mM imidazole; pH 7). Recombinantly-expressed FKBP12.6 (final concentration=250 nM) was added to the samples, in the absence or presence of different concentrations of JTV-519. After a 60-min incubation, samples were centrifuged at 100,000×g for 10 min, and washed twice in imidazole buffer. Samples were heated to 95° C., and size-fractionated using SDS-PAGE. Immunoblotting of the SR microsomes was performed, as previously described (Jayaraman et al., FK506 binding protein associated with the calcium release channel (ryanodine receptor). J. Biol. Chem., 267:9474-77, 1992), with anti-FKBP12.6 antibody (1:1,000) and anti-RyR2-5029 antibody (1:3,000). The figure demonstrates that JTV-519 enables FKBP12.6 to bind to: (A) PKA-phosphorylated RyR2 (partial binding at 100 nM; complete binding at 1000 nM) or (B) RyR2-S2809D mutant channels, which are constitutively PKA-phosphorylated RyR2 channels. (C-E) Single-channel studies showing increased open probability of RyR2 following PKA phosphorylation (D), as compared with PKA phosphorylation in the presence of the specific PKA inhibitor, PKI₅₋₂₄ (C). Single-channel function was normalized in PKA-phosphorylated RyR2 incubated with FKBP12.6 in the presence of JTV-519 (E). Channel openings are upward, the dash indicates the level of full openings (4 pA), and the letter ‘c’ indicates the closed state. Channels are shown at compressed (5 sec, upper tracing) and expanded (500 msec, lower tracing) time scales, and recordings are at 0 mV. Amplitude histograms (right) revealed increased activity and subconductance openings in PKA-phosphorylated RyR2, but not following treatment with JTV-519 and FKBP12.6. (F) Normalized plot of open probability as a function of cytosolic [Ca²⁺]. Incubation of PKA-phosphorylated RyR2 with FKBP12.6 in the presence of JTV-519 shifted the Ca²⁺-dependence of RyR2 activation towards the right, making it similar to the Ca²⁺-dependence of unphosphorylated channels.

### DETAILED DESCRIPTION OF THE INVENTION

As discussed above, catecholaminergic polymorphic ventricular tachycardia (CPVT) is an inherited disorder in individuals with structurally-normal hearts. It is characterized by stress-induced ventricular tachycardia, a lethal arrhythmia that may cause sudden cardiac death (SCD). Mutations in RyR2 channels, located on the sarcoplasmic reticulum (SR), have been linked to CPVT. To determine the molecular mechanism underlying the fatal cardiac arrhythmias in CPVT, the inventors studied CPVT-associated mutant RyR2 channels (e.g., S2246L, R2474S, N₄₁O₄K, R4497C).

All individuals with CPVT have exercise-induced cardiac arrhythmias. The inventors previously showed that exercise-induced arrhythmias and sudden death (in patients with CPVT) result from a reduced affinity of FKBP12.6 for RyR2. Herein, the inventors have demonstrated that exercise activates RyR2 as a result of phosphorylation by adenosine 3′,5′-monophosphate (cAMP)-dependent protein kinase (PKA). Mutant RyR2 channels, which had normal function in planar lipid bilayers under basal conditions, were more sensitive to activation by PKA phosphorylation—exhibiting increased activity (open probability) and prolonged open states, as compared with wild-type channels. In addition, PKA-phosphorylated mutant RyR2 channels were resistant to inhibition by Mg²⁺, a physiological inhibitor of the channel, and showed reduced binding to FKBP12.6 (which stabilizes the channel in the closed state). These findings indicate that, during exercise, when the RyR2 are PKA-phosphorylated, the mutant CPVT channels are more likely to open in the relaxation phase of the cardiac cycle (diastole), increasing the likelihood of arrhythmias triggered by SR Ca²⁺ leak. Since heart failure is a leading cause of death world wide, methods to repair the leak in RyR2 could prevent fatal arrhythmias in millions of patients world-wide.

The inventors have further demonstrated herein that JTV-519, a benzothiazepine derivative, prevents lethal ventricular arrhythmias in mice heterozygous for the FKBP12.6 gene. JTV-519 reduced the open probability of RyR2, isolated from FKBP12.6^{+/-} mice that died following exercise, by increasing the affinity of FKBP12.6 for PKA-phosphorylated RyR2. Moreover, JTV-519 normalized gating of CPVT-associated mutant RyR2 channels by increasing FKBP12.6 binding affinity. These data indicate that JTV-519 may prevent fatal ventricular arrhythmias by increasing PKBP12.6-RyR2 binding affinity.

Novel Methods of Treatment and Prevention

In accordance with the foregoing, the present invention provides a method for limiting or preventing a decrease in the level of RyR2-bound FKBP12.6 in cells of a subject. As used herein, “FKBP12.6” includes both an “FKBP12.6 protein” and an “FKBP12.6 analogue”. Unless otherwise indicated herein, “protein” shall include a protein, protein domain, polypeptide, or peptide, and any fragment thereof. An “FKBP12.6 analogue” is a functional variant of the FKBP12.6 protein, having FKBP12.6 biological activity, that has 60% or greater (preferably, 70% or greater) amino-acid-sequence homology with the FKBP12.6 protein. As further used herein, the term “FKBP12.6 biological activity” refers to the activity of a protein or peptide that demonstrates an ability to associate physically with, or bind with, unphosphorylated or non-hyperphosphorylated RyR2 (i.e., binding of approximately two fold, or, more preferably, approximately five fold, above the background binding of a negative control), under the conditions of the assays described herein, although affinity may be different from that of FKBP12.6.

In addition, as used herein, “RyR2” includes both an “RyR2 protein” and an “RyR2 analogue”. An “RyR2 analogue” is a functional variant of the RyR2 protein, having RyR2 biological activity, that has 60% or greater (preferably, 70% or greater) amino-acid-sequence homology with the RyR2 protein. The RyR2 of the present invention may be unphosphorylated, phosphorylated, or hyperphosphorylated. As further used herein, the term “RyR2 biological activity” refers to the activity of a protein or peptide that demonstrates an ability to associate physically with, or bind with, FKBP12.6 (i.e., binding of approximately two fold, or, more preferably, approximately five fold, above the background binding of a negative control), under the conditions of the assays described herein, although affinity may be different from that of RyR2.

As described above, the cardiac ryanodine receptor, RyR2, is a protein complex comprising four 565,000-dalton RyR2 proteins in association with four 12,000-dalton FKBP12.6 proteins. FK506 binding proteins (FKBPs) are cis-trans peptidyl-prolyl isomerases that are widely expressed, and serve a variety of cellular functions. FKBP12.6 protein is tightly bound to, and regulates the function of, RyR2. FKBP12.6 binds to the RyR2 channel, one molecule per RyR2 subunit, stabilizes RyR2-channel function, and facilitates coupled gating between neighboring RyR2 channels, thereby preventing aberrant activation of the channel during the resting phase of the cardiac cycle. Accordingly, as used herein, the term “RyR2-bound FKBP12.6” includes a molecule of an FKBP12.6 protein that is bound to an RyR2 protein subunit or a tetramer of FKBP12.6 that is in association with a tetramer of RyR2.

In accordance with the method of the present invention, a “decrease” in the level of RyR2-bound FKBP12.6 in cells of a subject refers to a detectable decrease, diminution, or reduction in the level of RyR2-bound FKBP12.6 in cells of the subject. Such a decrease is limited or prevented in cells of a subject when the decrease is in any way halted, hindered, impeded, obstructed, or reduced by the administration of JTV-519 (as described below), such that the level of RyR2-bound FKBP12.6 in cells of the subject is higher than it would otherwise be in the absence of JTV-519.

The level of RyR2-bound FKBP12.6 in a subject may be detected by standard assays and techniques, including those readily determined from the known art (e.g., immunological techniques, hybridization analysis, immunoprecipitation, Western-blot analysis, fluorescence imaging techniques, and/or radiation detection, etc.), as well as any assays and detection methods disclosed herein. For example, protein may be isolated and purified from cells of a subject using standard methods known in the art, including, without limitation, extraction from the cells (e.g., with a detergent that solubilizes the protein) where necessary, followed by affinity purification on a column, chromatography (e.g., FTLC and HPLC), immunoprecipitation (with an antibody), and precipitation (e.g., with isopropanol and a reagent such as Trizol). Isolation and purification of the protein may be followed by electrophoresis (e.g., on an SDS-polyacrylamide gel). A decrease in the level of RyR2-bound FKBP12.6 in a subject, or the limiting or prevention thereof, may be determined by comparing the amount of RyR2-bound FKBP12.6 detected prior to the administration of JTV-519 (in accordance with methods described below) with the amount detected a suitable time after administration of JTV-519.

In the method of the present invention, a decrease in the level of RyR2-bound FKBP12.6 in cells of a subject may be limited or prevented, for example, by inhibiting dissociation of FKBP12.6 and RyR2 in cells of the subject; by increasing binding between FKBP12.6 and RyR2 in cells of the subject; or by stabilizing the RyR2-FKBP12.6 complex in cells of a subject. As used herein, the term “inhibiting dissociation” includes blocking, decreasing, inhibiting, limiting, or preventing the physical dissociation or separation of an FKBP12.6 subunit from an RyR2 molecule in cells of the subject, and blocking, decreasing, inhibiting, limiting, or preventing the physical dissociation or separation of an RyR2 molecule from an FKBP12.6 subunit in cells of the subject. As further used herein, the term “increasing binding” includes enhancing, increasing, or improving the ability of phosphorylated RyR2 to associate physically with FKBP12.6 (e.g., binding of approximately two fold, or, more preferably, approximately five fold, above the background binding of a negative control) in cells of the subject, and enhancing, increasing, or improving the ability of FKBP12.6 to associate physically with phosphorylated RyR2 (e.g., binding of approximately two fold, or, more preferably, approximately five fold, above the background binding of a negative control) in cells of the subject. Additionally, in the method of the present invention, a decrease in the level of RyR2-bound FKBP12.6 in cells of a subject may be limited or prevented by directly decreasing the level of phosphorylated RyR2 in cells of the subject, or by indirectly decreasing the level of phosphorylated RyR2 in the cells (e.g., by targeting an enzyme (such as PKA) or another endogenous molecule that regulates or modulates the functions or levels of phosphorylated RyR2 in the cells). Preferably, the level of phosphorylated RyR2 in the cells is decreased by at least 10% in the method of the present invention. More preferably, the level of phosphorylated RyR2 is decreased by at least 20%.

In accordance with the method of the present invention, a decrease in the level of RyR2-bound FKBP12.6 is limited or prevented in a subject, particularly in cells of a subject. The subject of the present invention may be any animal, including amphibians, birds, fish, mammals, and marsupials, but is preferably a mammal (e.g., a human; a domestic animal, such as a cat, dog, monkey, mouse, or rat; or a commercial animal, such as a cow or pig). Additionally, the subject of the present invention is a candidate for exercise-induced cardiac arrhythmia. Exercise-induced cardiac arrhythmia is a heart condition (e.g., a ventricular fibrillation or ventricular tachycardia, including any that leads to sudden cardiac death) that develops during/after a subject has undergone physical exercise. A “candidate” for exercise-induced cardiac arrhythmia is a subject who is known to be, or is believed to be, or is suspected of being, at risk for developing cardiac arrhythmia during/after physical exercise. Examples of candidates for exercise-induced cardiac arrhythmia include, without limitation, an animal/person known to have catecholaminergic polymorphic ventricular tachycardia (CPVT); an animal/person suspected of having CPVT; and an animal/person who is known to be, or is believed to be, or is suspected of being, at risk for developing cardiac arrhythmia during/after physical exercise, and who is about to exercise, is currently exercising, or has just completed exercise. As discussed above, CPVT is an inherited disorder in individuals with structurally-normal hearts. It is characterized by stress-induced ventricular tachycardia—a lethal arrhythmia that may cause sudden cardiac death. In subjects with CPVT, physical exertion and/or stress induce bidirectional and/or polymorphic ventricular tachycardias that lead to sudden cardiac death (SCD) in the absence of detectable structural heart disease. Individuals with CPVT have ventricular arrhythmias when subjected to exercise, but do not develop arrhythmias at rest.

In the method of the present invention, the cells of a subject are preferably striated muscle cells. A striated muscle is a muscle in which the repeating units (sarcomeres) of the contractile myofibrils are arranged in registry throughout the cell, resulting in transverse or oblique striations that may be observed at the level of a light microscope. Examples of striated muscle cells include, without limitation, voluntary (skeletal) muscle cells and cardiac muscle cells. In a preferred embodiment, the cell used in the method of the present invention is a human cardiac muscle cell. As used herein, the term “cardiac muscle cell” includes cardiac muscle fibers, such as those found in the myocardium of the heart. Cardiac muscle fibers are composed of chains of contiguous heart-muscle cells, or cardiomyocytes, joined end to end at intercalated disks. These disks possess two kinds of cell junctions: expanded desmosomes extending along their transverse portions, and gap junctions, the largest of which lie along their longitudinal portions.

In the method of the present invention, a decrease in the level of RyR2-bound FKBP12.6 is limited or prevented in cells of a subject by administering JTV-519 to the subject; this would also permit contact between cells of the subject and JTV-519. JTV-519 (4-[3-(4-benzylpiperidin-1-yl)propionyl]-7-methoxy-2,3,4,5-tetrahydro-1,4-benzothiazepine monohydrochloride), also known as k201, is a derivative of 1,4-benzothiazepine, and a modulator of calcium-ion channels. In addition to regulating Ca²⁺ levels in myocardial cells, JTV-519 modulates the Na⁺ current and the inward-rectifier K⁺ current in guinea pig ventricular cells, and inhibits the delayed-rectifier K⁺ current in guinea pig atrial cells. FK506 and rapamycin are drugs that may be used to design other compounds that stabilize RyR2-FKBP12.6 binding in cells of a subject who is a candidate for exercise-induced cardiac arrhythmia. FK506 and rapamycin both dissociate FKBP12.6 from RyR2. It is possible to design and/or screen for compounds that are structurally related to these drugs, but have the opposite effects.

In the method of the present invention, JTV-519 may be administered to a subject by way of a therapeutic composition, comprising JTV-519 and a pharmaceutically-acceptable carrier. The pharmaceutically-acceptable carrier must be “acceptable” in the sense of being compatible with the other ingredients of the composition, and not deleterious to the recipient thereof. The pharmaceutically-acceptable carrier employed herein is selected from various organic or inorganic materials that are used as materials for pharmaceutical formulations, and which may be incorporated as analgesic agents, buffers, binders, disintegrants, diluents, emulsifiers, excipients, extenders, glidants, solubilizers, stabilizers, suspending agents, tonicity agents, vehicles, and viscosity-increasing agents. If necessary, pharmaceutical additives, such as antioxidants, aromatics, colorants, flavor-improving agents, preservatives, and sweeteners, may also be added. Examples of acceptable pharmaceutical carriers include carboxymethyl cellulose, crystalline cellulose, glycerin, gum arabic, lactose, magnesium stearate, methyl cellulose, powders, saline, sodium alginate, sucrose, starch, talc, and water, among others.

The pharmaceutical formulations of the present invention may be prepared by methods well-known in the pharmaceutical arts. For example, the JTV-519 may be brought into association with a carrier or diluent, as a suspension or solution. Optionally, one or more accessory ingredients (e.g., buffers, flavoring agents, surface active agents, and the like) also may be added. The choice of carrier will depend upon the route of administration.

JTV-519 may be administered to a subject by contacting target cells (e.g., cardiac muscle cells) in vivo in the subject with the JTV-519. JTV-519 may be contacted with (e.g., introduced into) cells of the subject using known techniques utilized for the introduction and administration of proteins, nucleic acids, and other drugs. Examples of methods for contacting the cells with (i.e., treating the cells with) JTV-519 include, without limitation, absorption, electroporation, immersion, injection, introduction, liposome delivery, transfection, transfusion, vectors, and other drug-delivery vehicles and methods. When the target cells are localized to a particular portion of a subject, it may be desirable to introduce the JTV-519 directly to the cells, by injection or by some other means (e.g., by introducing the JTV-519 into the blood or another body fluid). The target cells may be contained in heart tissue of a subject, and may be detected in heart tissue of the subject by standard detection methods readily determined from the known art, examples of which include, without limitation, immunological techniques (e.g., immunohistochemical staining), fluorescence imaging techniques, and microscopic techniques.

Additionally, the JTV-519 of the present invention may be administered to a human or animal subject by known procedures, including, without limitation, oral administration, parenteral administration, and transdermal administration. Preferably, the JTV-519 is administered parenterally, by epifascial, intracapsular, intracranial, intracutaneous, intrathecal, intramuscular, intraorbital, intraperitoneal, intraspinal, intrasternal, intravascular, intravenous, parenchymatous, subcutaneous, or sublingual injection, or by way of catheter. In one embodiment, the agent is administered to the subject by way of targeted delivery to cardiac muscle cells via a catheter inserted into the subject's heart.

For oral administration, a JTV-519 formulation may be presented as capsules, tablets, powders, granules, or as a suspension. The formulation may have conventional additives, such as lactose, mannitol, corn starch, or potato starch. The formulation also may be presented with binders, such as crystalline cellulose, cellulose derivatives, acacia, corn starch, or gelatins. Additionally, the formulation may be presented with disintegrators, such as corn starch, potato starch, or sodium carboxymethylcellulose. The formulation also may be presented with dibasic calcium phosphate anhydrous or sodium starch glycolate. Finally, the formulation may be presented with lubricants, such as talc or magnesium stearate.

For parenteral administration (i.e., administration by injection through a route other than the alimentary canal), JTV-519 may be combined with a sterile aqueous solution that is preferably isotonic with the blood of the subject. Such a formulation may be prepared by dissolving a solid active ingredient in water containing physiologically-compatible substances, such as sodium chloride, glycine, and the like, and having a buffered pH compatible with physiological conditions, so as to produce an aqueous solution, then rendering said solution sterile. The formulation may be presented in unit or multi-dose containers, such as sealed ampoules or vials. The formulation may be delivered by any mode of injection, including, without limitation, epifascial, intracapsular, intracranial, intracutaneous, intrathecal, intramuscular, intraorbital, intraperitoneal, intraspinal, intrasternal, intravascular, intravenous, parenchymatous, subcutaneous, or sublingual, or by way of catheter into the subject's heart.

For transdermal administration, JTV-519 may be combined with skin penetration enhancers, such as propylene glycol, polyethylene glycol, isopropanol, ethanol, oleic acid, N-methylpyrrolidone, and the like, which increase the permeability of the skin to the JTV-519, and permit the JTV-519 to penetrate through the skin and into the bloodstream. The JTV-519/enhancer composition also may be further combined with a polymeric substance, such as ethylcellulose, hydroxypropyl cellulose, ethylene/vinylacetate, polyvinyl pyrrolidone, and the like, to provide the composition in gel form, which may be dissolved in a solvent, such as methylene chloride, evaporated to the desired viscosity, and then applied to backing material to provide a patch.

In accordance with the method of the present invention, JTV-519 may be administered to the subject (and JTV-519 may be contacted with cells of the subject) in an amount effective to limit or prevent a decrease in the level of RyR2-bound FKBP12.6 in the subject, particularly in cells of the subject. This amount may be readily determined by the skilled artisan, based upon known procedures, including analysis of titration curves established in vivo, and methods and assays disclosed herein. A suitable amount of JTV-519 effective to limit or prevent a decrease in the level of RyR2-bound FKBP12.6 in the subject may range from about 5 mg/kg/day to about 20 mg/kg/day, and/or may be an amount sufficient to achieve plasma levels ranging from about 300 ng/ml to about 1000 ng/ml. Preferably, the amount of JTV-519 ranges from about 10 mg/kg/day to about 20 mg/kg/day.

In one embodiment of the present invention, the subject has not yet developed exercise-induced cardiac arrhythmia. In this case, the amount of JTV-519 effective to limit or prevent a decrease in the level of RyR2-bound FKBP12.6 in the subject may be an amount of JTV-519 effective to prevent exercise-induced cardiac arrhythmia in the subject. Cardiac arrhythmia is a disturbance of the electrical activity of the heart that manifests as an abnormality in heart rate or heart rhythm. As used herein, an amount of JTV-519 “effective to prevent exercise-induced cardiac arrhythmia” includes an amount of JTV-519 effective to prevent the development of the clinical impairment or symptoms of the exercise-induced cardiac arrhythmia (e.g., palpitations, fainting, ventricular fibrillation, ventricular tachycardia, and sudden cardiac death). The amount of JTV-519 effective to prevent exercise-induced cardiac arrhythmia in a subject will vary depending upon the particular factors of each case, including the type of exercise-induced cardiac arrhythmia, the subject's weight, the severity of the subject's condition, and the mode of administration of the JTV-519. This amount may be readily determined by the skilled artisan, based upon known procedures, including clinical trials, and methods disclosed herein. In a preferred embodiment, the amount of JTV-519 effective to prevent the exercise-induced cardiac arrhythmia is an amount of JTV-519 effective to prevent exercise-induced sudden cardiac death in the subject. In another preferred embodiment, the JTV-519 prevents exercise-induced cardiac arrhythmia and exercise-induced sudden cardiac death in the subject.

Because of its ability to stabilize RyR2-bound FKBP12.6, and maintain and restore balance in the context of dynamic PKA phosphorylation and dephosphorylation of RyR2, JTV-519 may also be useful in treating a subject who has already started to experience clinical symptoms of exercise-induced cardiac arrhythmia. If the symptoms of arrhythmia are observed in the subject early enough, JTV-519 might be effective in limiting or preventing a further decrease in the level of RyR2-bound FKBP12.6 in the subject.

Accordingly, in still another embodiment of the present invention, the subject has been exercising, or is currently exercising, and has developed exercise-induced cardiac arrhythmia. In this case, the amount of JTV-519 effective to limit or prevent a decrease in the level of RyR2-bound FKBP12.6 in the subject may be an amount of JTV-519 effective to treat exercise-induced cardiac arrhythmia in the subject. As used herein, an amount of JTV-519 “effective to treat exercise-induced cardiac arrhythmia” includes an amount of JTV-519 effective to alleviate or ameliorate the clinical impairment or symptoms of the exercise-induced cardiac arrhythmia (e.g., palpitations, fainting, ventricular fibrillation, ventricular tachycardia, and sudden cardiac death). The amount of JTV-519 effective to treat exercise-induced cardiac arrhythmia in a subject will vary depending upon the particular factors of each case, including the type of exercise-induced cardiac arrhythmia, the subject's weight, the severity of the subject's condition, and the mode of administration of the JTV-519. This amount may be readily determined by the skilled artisan, based upon known procedures, including clinical trials, and methods disclosed herein. In a preferred embodiment, the JTV-519 treats exercise-induced cardiac arrhythmia in the subject.

The present invention further provides a method for treating exercise-induced cardiac arrhythmia in a subject. The method comprises administering JTV-519 to the subject in an amount effective to treat exercise-induced cardiac arrhythmia in the subject. A suitable amount of JTV-519 effective to treat exercise-induced cardiac arrhythmia in the subject may range from about 5 mg/kg/day to about 20 mg/kg/day, and/or may be an amount sufficient to achieve plasma levels ranging from about 300 ng/ml to about 1000 ng/ml. The present invention also provides a method for preventing exercise-induced cardiac arrhythmia in a subject. The method comprises administering JTV-519 to the subject in an amount effective to prevent exercise-induced cardiac arrhythmia in the subject. A suitable amount of JTV-519 effective to prevent exercise-induced cardiac arrhythmia in the subject may range from about 5 mg/kg/day to about 20 mg/kg/day, and/or may be an amount sufficient to achieve plasma levels ranging from about 300 ng/ml to about 1000 ng/ml. Additionally, the present invention provides a method for preventing exercise-induced sudden cardiac death in a subject. The method comprises administering JTV-519 to the subject in an amount effective to prevent exercise-induced sudden cardiac death in the subject. A suitable amount of JTV-519 effective to prevent exercise-induced sudden cardiac death in the subject may range from about 5 mg/kg/day to about 20 mg/kg/day, and/or may be an amount sufficient to achieve plasma levels ranging from about 300 ng/ml to about 1000 ng/ml.

In various embodiments of the above-described methods, the exercise-induced cardiac arrhythmia in the subject is associated with VT. In preferred embodiments, the VT is CPVT. In other embodiments of these methods, the subject is a candidate for exercise-induced cardiac arrhythmia, including candidates for exercise-induced sudden cardiac death.

In view of the foregoing methods, the present invention also provides use of JTV-519 in a method for limiting or preventing a decrease in the level of RyR2-bound FKBP12.6 in a subject who is a candidate for exercise-induced cardiac arrhythmia. The present invention also provides use of JTV-519 in a method for treating or preventing exercise-induced cardiac arrhythmia in a subject. Furthermore, the present invention provides use of JTV-519 in a method for preventing exercise-induced sudden cardiac death in a subject.

As discussed above and presented herein, the inventors' data show that protein kinase A (PKA) phosphorylation of the cardiac ryanodine receptor, RyR2, on serine 2809 activates the channel by releasing the FK506 binding protein, FKBP12.6. In failing hearts (including human hearts and animal models of heart failure), RyR2 is PKA-hyperphosphorylated, resulting in defective channels that have decreased amounts of bound FKBP12.6, and have increased sensitivity to calcium-induced activation. The net result of these changes is that the RyR2 channels are “leaky”. These channel leaks can result in a depletion of intracellular stores of calcium to such an extent that there is no longer enough calcium in the sarcoplasmic reticulum (SR) to provide a strong stimulus for muscle contraction. This results in weak contraction of heart muscle. As a second consequence of the channel leaks, RyR2 channels release calcium during the resting phase of the heart cycle known as “diastole”. This release of calcium during diastole can trigger the fatal arrhythmias of the hearts (e.g., ventricular tachycardia and ventricular fibrillation) that cause sudden cardiac death (SCD).

The inventors have also shown that treatment of heart failure with a mechanical pumping device, referred to as a left ventricular assist device (LVAD), which puts the heart at rest and restores normalized function, is associated with a reduction in the PKA hyperphosphorylation of RyR2, and normalized function of the channel. Furthermore, the inventors have shown that treatment of dogs (who have pacing-induced heart failure) with beta-adrenergic blockers (beta blockers) reverses the PKA hyperphosphorylation of RyR2. Beta blockers inhibit the pathway that activates PKA. The conclusion which may be drawn from the results of the inventors' work is that PKA phosphorylation of RyR2 increases the activity of the channel, resulting in the release of more calcium into the cell for a given trigger (activator) of the channel.

As further disclosed herein, the inventors have established that exercise-induced sudden cardiac death is associated with an increase in phosphorylation of RyR2 proteins (particularly CPVT-associated RyR2 mutant proteins) and a decrease in the level of RyR2-bound FKBP12.6. It is possible to use this mechanism to design effective drugs for preventing exercise-induced sudden cardiac death. A candidate agent having the ability to limit or prevent a decrease in the level of RyR2-bound FKBP12.6 may, as a consequence of this limiting or preventive activity, have an effect on an RyR2-associated biological event, thereby preventing exercise-induced sudden cardiac death.

Accordingly, the present invention further provides a method for identifying an agent for use in preventing exercise-induced sudden cardiac death. The method comprises the steps of: (a) obtaining or generating a culture of cells containing RyR2; (b) contacting the cells with a candidate agent; (c) exposing the cells to one or more conditions known to increase phosphorylation of RyR2 in cells; and (d) determining if the agent limits or prevents a decrease in the level of RyR2-bound FKBP12.6 in the cells. As used herein, an “agent” shall include a protein, polypeptide, peptide, nucleic acid (including DNA or RNA), antibody, Fab fragment, F(ab′)₂ fragment, molecule, compound, antibiotic, drug, and any combination(s) thereof. An agent that limits or prevents a decrease in the level of RyR2-bound FKBP12.6 may be either natural or synthetic, and may be an agent reactive with (i.e., an agent that has affinity for, binds to, or is directed against) RyR2 and/or FKBP12.6. As further used herein, a cell “containing RyR2” is a cell (preferably, a cardiac muscle cell) in which RyR2, or a derivative or homologue thereof, is naturally expressed or naturally occurs. Conditions known to increase phosphorylation of RyR2 in cells include, without limitation, PKA.

In the method of the present invention, cells may be contacted with a candidate agent by any of the standard methods of effecting contact between drugs/agents and cells, including any modes of introduction and administration described herein. The level of RyR2-bound FKBP12.6 in the cell may be measured or detected by known procedures, including any of the methods, molecular procedures, and assays known to one of skill in the art or described herein. In one embodiment of the present invention, the agent limits or prevents a decrease in the level of RyR2-bound FKBP12.6 in the cells.

As disclosed herein, RyR2 has been implicated in a number of biological events in striated muscle cells. For example, it has been shown that RyR2 channels play an important role in EC coupling and contractility in cardiac muscle cells. Therefore, it is clear that preventive drugs designed to limit or prevent a decrease in the level of RyR2-bound FKBP12.6 in cells, particularly cardiac muscle cells, may be useful in the regulation of a number of RyR2-associated biological events, including EC coupling and contractility. Thus, once the candidate agent of the present invention has been screened, and has been determined to have a suitable limiting or preventive effect on decreasing levels of RyR2-bound FKBP12.6, it may be evaluated for its effect on EC coupling and contractility in cells, particularly cardiac muscle cells. It is expected that the preventive agent of the present invention will be useful for preventing exercise-induced sudden cardiac death.

Accordingly, the method of the present invention may further comprise the steps of: (e) contacting the candidate agent with a culture of cells containing RyR2; and (f) determining if the agent has an effect on an RyR2-associated biological event in the cells. As used herein, an “RyR2-associated biological event” includes a biochemical or physiological process in which RyR2 levels or activity have been implicated. As disclosed herein, examples of RyR2-associated biological events include, without limitation, EC coupling and contractility in cardiac muscle cells. According to this method of the present invention, a candidate agent may be contacted with one or more cells (preferably, cardiac muscle cells) in vitro. For example, a culture of the cells may be incubated with a preparation containing the candidate agent. The candidate agent's effect on an RyR2-associated biological event then may be assessed by any biological assays or methods known in the art, including immunoblotting, single-channel recordings and any others disclosed herein.

The present invention is further directed to an agent identified by the above-described identification method, as well as a pharmaceutical composition comprising the agent and a pharmaceutically-acceptable carrier. The agent may be useful for preventing exercise-induced sudden cardiac death in a subject, and for treating or preventing other RyR2-associated conditions. As used herein, an “RyR2-associated condition” is a condition, disease, or disorder in which RyR2 level or activity has been implicated, and includes an RyR2-associated biological event. The RyR2-associated condition may be treated or prevented in the subject by administering to the subject an amount of the agent effective to treat or prevent the RyR2-associated condition in the subject. This amount may be readily determined by one skilled in the art. In one embodiment, the present invention provides a method for preventing exercise-induced sudden cardiac death in a subject, by administering the agent to the subject in an amount effective to prevent the exercise-induced sudden cardiac death in the subject.

The present invention also provides an in vivo method for identifying an agent for use in preventing exercise-induced sudden cardiac death. The method comprises the steps of: (a) obtaining or generating an animal containing RyR2; (b) administering a candidate agent to the animal; (c) exposing the animal to one or more conditions known to increase phosphorylation of RyR2 in cells; and (d) determining if the agent limits or prevents a decrease in the level of RyR2-bound FKBP12.6 in the animal. The method may further comprise the steps of: (e) administering the agent to an animal containing RyR2; and (f) determining if the agent has an effect on an RyR2-associated biological event in the animal. Also provided is an agent identified by this method; a pharmaceutical composition comprising this agent; and a method for preventing exercise-induced sudden cardiac death in a subject, by administering this agent to the subject in an amount effective to prevent the exercise-induced sudden cardiac death in the subject.

The inventors' work has demonstrated that compounds which block PKA activation would be expected to reduce the activation of the RyR2 channel, resulting in less release of calcium into the cell. Compounds that bind to the RyR2 channel at the FKBP12.6 binding site, but do not come off the channel when the channel is phosphorylated by PKA, would also be expected to decrease the activity of the channel in response to PKA activation or other triggers that activate the RyR2 channel. Such compounds would also result in less calcium release into the cell. In view of these findings, the present invention further provides additional assays for identifying agents that may be useful in preventing exercise-induced sudden cardiac death, in that they block or inhibit activation of RyR2.

By way of example, the diagnostic assays of the present invention may screen for the release of calcium into cells via the RyR2 channel, using calcium-sensitive fluorescent dyes (e.g., Fluo-3, Fura-2, and the like). Cells may be loaded with the fluorescent dye of choice, then stimulated with RyR2 activators to determine whether or not compounds added to the cell reduce the calcium-dependent fluorescent signal (Brillantes et al., Stabilization of calcium release channel (ryanodine receptor) function by FK506-binding protein. Cell, 77:513-23, 1994; Gillo et al., Calcium entry during induced differentiation in murine erythroleukemia cells. Blood, 81:783-92, 1993; Jayaraman et al., Regulation of the inositol 1,4,5-trisphosphate receptor by tyrosine phosphorylation. Science, 272:1492-94, 1996). Calcium-dependent fluorescent signals may be monitored with a photomultiplier tube, and analyzed with appropriate software, as previously described (Brillantes et al., Stabilization of calcium release channel (ryanodine receptor) function by FK506-binding protein. Cell, 77:513-23, 1994; Gillo et al., Calcium entry during induced differentiation in murine erythroleukemia cells. Blood, 81:783-92, 1993; Jayaraman et al., Regulation of the inositol 1,4,5-trisphosphate receptor by tyrosine phosphorylation. Science, 272:1492-94, 1996). This assay can easily be automated to screen large numbers of compounds using multiwell dishes.

To identify compounds that inhibit the PKA-dependent activation of RyR2-mediated intracellular calcium release, an assay may involve the expression of recombinant RyR2 channels in a heterologous expression system, such as Sf9, HEK293, or CHO cells (Brillantes et al., Stabilization of calcium release channel (ryanodine receptor) function by FK506-binding protein. Cell, 77:513-23, 1994). RyR2 could also be co-expressed with beta-adrenergic receptors. This would permit assessment of the effect of compounds on RyR2 activation, in response to addition of beta-adrenergic receptor agonists.

The level of PKA phosphorylation of RyR2 which correlates with the degree of heart failure may also be assayed, and then used to determine the efficacy of compounds designed to block the PKA phosphorylation of the RyR2 channel. Such an assay may be based on the use of antibodies that are specific for the RyR2 protein. For example, the RyR2-channel protein may be immunoprecipitated, and then back-phosphorylated with PKA and [γ³²P]-ATP. The amount of radioactive [³²P] label that is transferred to the RyR2 protein may be then measured using a phosphorimager (Marx et al., PKA phosphorylation dissociates FKBP12.6 from the calcium release channel (ryanodine receptor): defective regulation in failing hearts. Cell, 101:365-76, 2000).

Another assay of the present invention involves use of a phosphoepitope-specific antibody that detects RyR2 that is PKA phosphorylated on Ser 2809. Immunoblotting with such an antibody can be used to assess efficacy of therapy for heart failure and cardiac arrhythmias. Additionally, RyR2 S2809A and RyR2 S2809D knock-in mice may be used to assess efficacy of therapy for heart failure and cardiac arrhythmias. Such mice further provide evidence that PKA hyperphosphorylation of RyR2 is a contributing factor in heart failure and cardiac arrhythmias, by showing that the RyR2 S2809A mutation inhibits heart failure and arrhythmias, and that the RyR2 S2809D mutation worsens heart failure and arrhythmias.

Novel Pathways of Chemical Synthesis

1,4-benzothiazepine derivatives are important building blocks in the preparation of biologically-active molecules, including JTV-519. The inventors have developed a novel process for preparing 1,4-benzothiazepine intermediate compounds, such as 7-methoxy-2,3,4,5-tetrahydro-1,4-benzothiazepine. The inventors' process utilizes readily-available and inexpensive starting materials, and provides high yields of key 1,4-benzothiazepine intermediates.

In the early 1990s, Kaneko et al. (U.S. Pat. No. 5,416,066; WO 92/12148; JP4230681) disclosed that JTV-519 could be prepared by reacting 7-methoxy-2,3,4,5-tetrahydro-1,4-benzothiazepine (a 1,4-benzothiazepine intermediate) with acryloyl chloride, and then reacting the resulting product with 4-benzyl piperidine.

Two processes for the preparation of 7-methoxy-2,3,4,5-tetrahydro-1,4-benzothiazepine and similar compounds have been previously reported in the literature. The first process, disclosed by Kaneko et al. (U.S. Pat. No. 5,416,066), involved a synthetic route of six steps that started with 2,5-dihydroxybenzoic acid. In this process, 2,5-dihydroxybenzoic acid was selectively methylated with dimethyl sulfate. The resulting compound was then reacted with dimethylthiocarbamoyl chloride for 20 h, and then subjected to high temperature (270° C.) for 9 h. The product of this step was refluxed with sodium methoxide in methanol for 20 h. The product of the reflux step was then reacted with 2-chloroethylamine, under basic conditions and at a high temperature, to produce a cyclized amide. The cyclized amide was reduced with LiAlH₄ to yield 7-methoxy-2,3,4,5-tetrahydro-1,4-benzothiazepine (a 1,4-benzothiazepine intermediate).

The second process for the preparation of 7-methoxy-2,3,4,5-tetrahydro-1,4-benzothiazepine was disclosed by Hitoshi in a Japanese patent (JP 10045706). This process started with 2-bromo-5-methoxy benzaldehyde. The bromide was substituted with NaSMe, and the resulting product was oxidized with chlorine, followed by reflux in water, to yield disulfide dialdehyde. The dialdehyde was treated with 2-chloroethylamine, and the resulting product was reduced with a reducing agent, such as NaBH₄. The resulting compound was cyclized to give 7-methoxy-2,3,4,5-tetrahydro-1,4-benzothiazepine.

Initially, the inventors attempted to prepare the 1,4-benzothiazepine intermediate, 7-methoxy-2,3,4,5-tetrahydro-1,4-benzothiazepine, using the methods described above. However, they found that the first process, described by Kaneko et al. (U.S. Pat. No. 5,416,066), involved synthetic steps of high temperature and long reaction time. Additionally, the inventors discovered that the thio group in the third thiolated intermediate was easily oxidized by air to a disulfide compound, making it impossible to synthesize the subsequent cyclized product. The inventors also determined that the process described by Hitoshi (JP 10045706) involved Cl₂, and that another patented method for the preparation of the first intermediate, apart from the substitution of bromide with NaSMe, had to be used.

To overcome the foregoing problems, the inventors developed a novel process for making 7-methoxy-2,3,4,5-tetrahydro-1,4-benzothiazepine from readily-available and inexpensive starting materials. The inventors' process simplifies isolation and purification steps, and can be used to prepare various 1,4-benzothiazepine intermediates, including 7-methoxy-2,3,4,5-tetrahydro-1,4-benzothiazepine and other compounds having the general structure shown in formula:

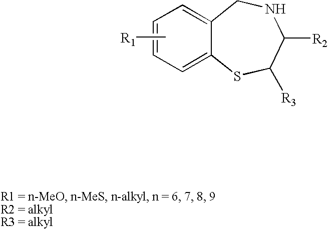

This process may also be used to prepare JTV-519.

Accordingly, in view of the foregoing, the present invention provides a method for the synthesis of a compound of a compound having formula:

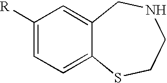

wherein R=OR′, SR′, NR′, alkyl, or halide and R′=alkyl, aryl, or H, and wherein R can be at position 2, 3, 4, or 5, said method comprising the steps of:

(a) treating a compound having formula:

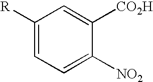

wherein R is as defined above, with a reducing agent, in the presence of an optional catalyst, to form a compound having formula:

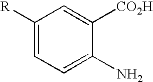

wherein R is as defined above;

(b) treating the compound formed in step (a) with a diazotizing agent and a disulfide, to form a compound having formula:

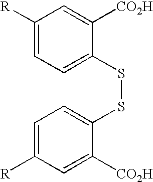

wherein R is as defined above;

(c) treating the compound formed in step (b) with a chloride and a chloroethylamine, to form a compound having formula:

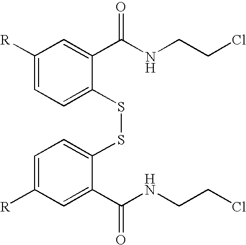

wherein R is as defined above;

(d) treating the compound formed in step (c) with a reducing agent and a base, in the presence of tetrahydrolate, to form a compound having formula:

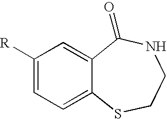

wherein R is as defined above; and

(e) treating the compound formed in step (d) with a reducing agent, to form a compound having formula:

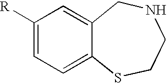

wherein R is as defined above.

In accordance with the method of the present invention, the reducing agent in step (a) may be H₂. Additionally, the diazotizing agent in step (b) may be NaNO₂, and the disulfide in step (b) may be Na₂S₂. Furthermore, the chloride in step (c) may be SOCl₂. The reducing agent in step (d) may be trimethylphosphine (PMe₃), while the base in step (d) is triethyl amine. In another embodiment, the reducing agent in step (e) is LiAlH₄.

The present invention further provides a method for the synthesis of a compound of having formula:

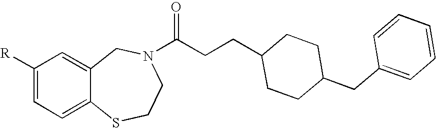

wherein R=OR′, SR′, NR′, alkyl, or halide and R′=alkyl, aryl, or H, and wherein R can be at position 2, 3, 4, or 5, said method comprising the step of:

(a) treating a compound having formula:

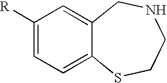

wherein R is as defined above, with 3-bromopropionic chloride and a compound having formula:

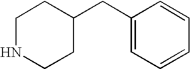

to form a compound having formula:

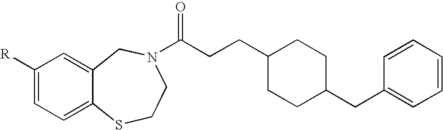

wherein R is as defined above.

By way of example, a compound having the formula:

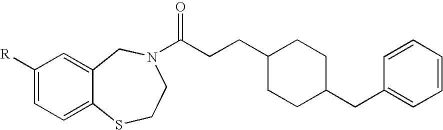

wherein R=OR′, SR′, NR′, alkyl, or halide and R′=alkyl, aryl, or H, and wherein R can be at position 2, 3, 4, or 5, may be synthesized as follows:

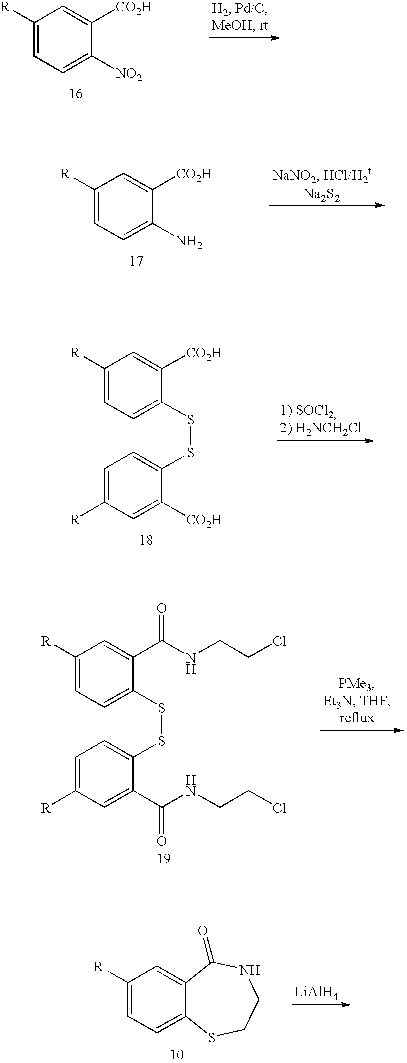

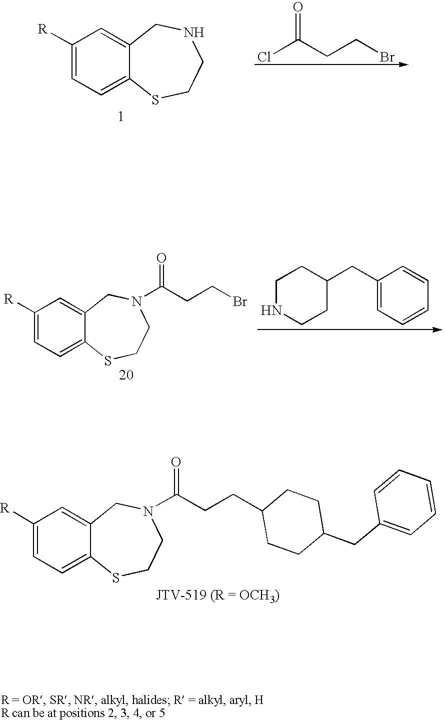

The method of the present invention further provides a method for the synthesis of a compound having formula:

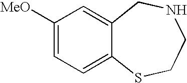

said method comprising the steps of:

(a) treating a compound having formula:

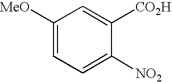

with a reducing agent, in the presence of an optional catalyst, to form a compound having formula:

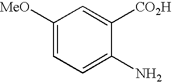

(b) treating the compound formed in step (a) with a diazotizing agent and a disulfide, to form a compound having formula:

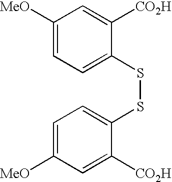

(c) treating the compound formed in step (b) with a chloride and a chloroethylamine, to form a compound having formula:

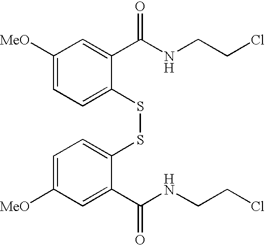

(d) treating the compound formed in step (c) with a reducing agent and a base, in the presence of tetrahydrolate, to form a compound having formula:

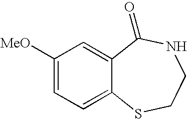

(e) treating the compound formed in step (d) with a reducing agent, to form a compound having formula:

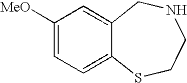

The present invention also provides a method for the synthesis of a compound having formula:

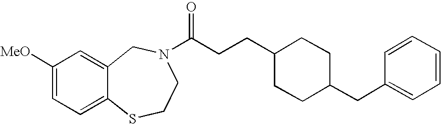

said method comprising the step of:

(a) treating a compound having formula:

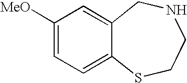

with 3-bromopropionic chloride and a compound having formula:

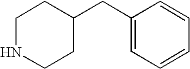

to form a compound having formula:

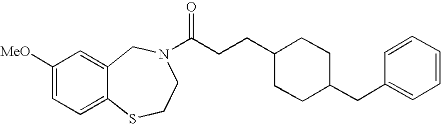

By way of example, and as shown in Example 7 and Scheme 1 below, 7-methoxy-2,3,4,5-tetrahydro-1,4-benzothiazepine may be prepared from 2-nitro-5-methoxybenzoic acid as follows. The nitro group of 2-nitro-5-methoxybenzoic acid is reduced, using H₂ with Pd/C as a catalyst, to give 2-amino-5-methoxybenzoic acid. 2-amino-5-methoxybenzoic acid may be diazotized with NaNO₂, and then treated with Na₂S₂, to provide a stable disulfide compound. Without further purification, the stable disulfide compound may be treated with SOCl₂, and then reacted with 2-chloroethylamine, in the presence of Et₃N, to give an amide. The amide compound may then be converted to a cyclized compound via a one-pot procedure, as follows. A reducing reagent (such as trimethylphosphine or triphenylphosphine) and a base (such as triethylamine) may be added to a solution of the amide compound in THF (tetrahydrofolate). The resulting reaction mixture may then be refluxed for 3 h. The reducing agent (trimethylphosphine or triphenylphine) cleaves the disulfide (S—S) to its monosulfide (—S), which, in situ, undergoes intramolecular cyclization with the chloride to yield a cyclized amide. The cyclized amide may then be reduced with LiAlH₄ to yield the 1,4-benzothiazepine intermediate, 7-methoxy-2,3,4,5-tetrahydro-1,4-benzothiazepine. JTV-519 may then be prepared from 7-methoxy-2,3,4,5-tetrahydro-1,4-benzothiazepine by reacting the 7-methoxy-2,3,4,5-tetrahydro-1,4-benzothiazepine with 3-bromopropionic chloride, and then reacting the resulting compound with 4-benzyl piperidine.

By way of example, and as shown in Example 8 and Scheme 2 below, radio-labeled JTV-519 may be prepared as follows. JTV-519 may be demethylated at the phenyl ring using BBr₃. The resulting phenol compound may then be re-methylated with a radio-labeled methylating agent (such as ³H-dimethyl sulfate) in the presence of a base (such as NaH) to provide ³H-labeled JTV-519.

The present invention further provides a composition, comprising radio-labeled JTV-519. Labeling of JTV-519 may be accomplished using one of a variety of different radioactive labels known in the art. The radioactive label of the present invention may be, for example, a radioisotope. The radioisotope may be any isotope that emits detectable radiation, including, without limitation, ³⁵S, ³²P, ¹²⁵I, ³H, or ¹⁴C. Radioactivity emitted by the radioisotope can be detected by techniques well known in the art. For example, gamma emission from the radioisotope may be detected using gamma imaging techniques, particularly scintigraphic imaging.

The present invention is described in the following Examples, which are set forth to aid in the understanding of the invention, and should not be construed to limit in any way the scope of the invention as defined in the claims which follow thereafter.

### EXAMPLES

### Example 1

### FKBP12.6-Deficient Mice

FKBP12.6-deficient mice were generated, as previously described (Wehrens et al., FKBP12.6 deficiency and defective calcium release channel (ryanodine receptor) function linked to exercise-induced sudden cardiac death. Cell, 113:829-40, 2003). Briefly, mouse genomic λ-phage clones for the murine orthologue of the human FK506 binding protein 12.6 (FKBP12.6) were isolated from a DBA/1lacJ library, using a full-length murine cDNA probe. The targeting vector was designed to delete exons 3 and 4, which contain the entire coding sequences for murine FKBP12.6 (Bennett et al., Identification and characterization of the murine FK506 binding protein (FKBP) 12.6 gene. Mamm. Genome, 9:1069-71, 1998), by replacing 3.5 kb of murine genomic DNA with a PGK-neo selectable marker. A 5.0-kb 5′ fragment and a 1.9-kb 3′ fragment were cloned into pJNS2, a backbone vector with PGK-neo and PGK-TK cassettes. The DBA/lacJ embryonic stem (ES) cells were grown and transfected, using established protocols. Targeted ES cells were first screened by Southern analysis, and 5 positive ES cell lines were analyzed by PCR to confirm homologous recombination. Male chimeras were bred to DBA/1lacJ females, and germline offspring identified by brown coat color. Germline offspring were genotyped using 5′ Southern analysis. Positive FKBP12.6^{+/-} males and females were intercrossed, and offspring resulted in FKBP12.6^{-/-} mice at approximately 25% frequency. FKBP12.6^{-/-} mice were fertile.

All studies performed with FKBP12.6^{-/-} mice used age- and sex-matched FKBP12.6^{+/+} mice as controls. No differences were observed between FKBP12.6^{-/-} mice raised on the following backgrounds: DBA/C57BL6 mixed, pure DBA, and pure C57BL6.

### Example 2

### Telemetry Recording and Exercise Testing in Mice

FKBP12.6^{+/+} and FKBP12.6^{-/-} mice were maintained and studied according to protocols approved by the Institutional Animal Care and Use Committee of Columbia University. Mice were anaesthetized using 2.5% isoflurane inhalation anesthesia. ECG radiotelemetry recordings of ambulatory animals were obtained >7 days after intraperitoneal implantation (Data Sciences International, St. Paul, Minn.) (Wehrens et al., FKBP12.6 deficiency and defective calcium release channel (ryanodine receptor) function linked to exercise-induced sudden cardiac death. Cell, 113:829-40, 2003). For stress tests, mice were exercised on an inclined treadmill until exhaustion, and then intraperitoneally injected with epinephrine (0.5-2.0 mg/kg) (Wehrens et al., FKBP12.6 deficiency and defective calcium release channel (ryanodine receptor) function linked to exercise-induced sudden cardiac death. Cell, 113:829-40, 2003). Resting heart rates of ambulatory animals were averaged over 4 h.

### Example 3

### Expression of Wild-Type and RyR2-S2809D Mutants

Mutagenesis of the PKA target site on RyR2 (RyR2-S2809D) was performed, as previously described (Wehrens et al., FKBP12.6 deficiency and defective calcium release channel (ryanodine receptor) function linked to exercise-induced sudden cardiac death. Cell, 113:829-40, 2003). HEK293 cells were co-transfected with 20 μg of RyR2 wild-type (WT) or mutant cDNA, and with 5 μg of FKBP12.6 cDNA, using Ca²⁺ phosphate precipitation. Vesicles containing RyR2 channels were prepared, as previously described (Wehrens et al., FKBP12.6 deficiency and defective calcium release channel (ryanodine receptor) function linked to exercise-induced sudden cardiac death. Cell, 113:829-40, 2003).

### Example 4

### RyR2 PKA Phosphorylation and FKBP12.6 Binding

Cardiac SR membranes were prepared, as previously described (Marx et al., PKA phosphorylation dissociates FKBP12.6 from the calcium release channel (ryanodine receptor): defective regulation in failing hearts. Cell, 101:365-76, 2000; Kaftan et al., Effects of rapamycin on ryanodine receptor/Ca⁽²⁺⁾-release channels from cardiac muscle. Circ. Res., 78:990-97, 1996). ³⁵S-labelled FKBP12.6 was generated using the TNT™ Quick Coupled Transcription/Translation system from Promega (Madison, Wis.). [³H] ryanodine binding was used to quantify RyR2 levels. 100 μg of microsomes were diluted in 100 μl of 10-mM imidazole buffer (pH 6.8), incubated with 250-nM (final concentration) [³⁵S]-FKBP12.6 at 37° C. for 60 min, then quenched with 500 μl of ice-cold imidazole buffer. Samples were centrifuged at 100,000 g for 10 min, and washed three times in imidazole buffer. The amount of bound [³⁵S]-FKBP12.6 was determined by liquid scintillation counting of the pellet.

### Example 5

### Immunoblots

Immunoblotting of microsomes (50 μg) was performed as described, with anti-FKBP12/12.6 (1:1,000), anti-RyR (5029; 1:3,000) (Jayaraman et al., FK506 binding protein associated with the calcium release channel (ryanodine receptor). J. Biol. Chem., 267:9474-77, 1992), or anti-phosphORyR2 (P2809; 1:5,000) for 1 h at room temperature (Reiken et al., Beta-blockers restore calcium release channel function and improve cardiac muscle performance in human heart failure. Circulation, 107:2459-66, 2003). The P2809-phosphoepitope-specific anti-RyR2 antibody is an affinity-purified polyclonal rabbit antibody, custom-made by Zymed Laboratories (San Francisco, Calif.) using the peptide, CRTRRI-(pS)-QTSQ, which corresponds to RyR2 PKA-phosphorylated at Ser²⁸⁰⁹. After incubation with HRP-labeled anti-rabbit IgG (1:5,000 dilution; Transduction Laboratories, Lexington, Ky.), the blots were developed using ECL (Amersham Pharmacia, Piscataway, N.J.).

### Example 6

### Single-Channel Recordings

Single-channel recordings of native RyR2 from mouse hearts, or recombinant RyR2, were acquired under voltage-clamp conditions at 0 mV, as previously described (Marx et al., PKA phosphorylation dissociates FKBP12.6 from the calcium release channel (ryanodine receptor): defective regulation in failing hearts. Cell, 101:365-76, 2000). Symmetric solutions used for channel recordings were: trans compartment —HEPES, 250 mmol/L; Ba(OH)₂, 53 mmol/L (in some experiments, Ba(OH)₂ was replaced by Ca(OH)₂); pH 7.35; and cis compartment —HEPES, 250 mmol/L; Tris-base, 125 mmol/L; EGTA, 1.0 mmol/L; and CaCl₂, 0.5 mmol/L; pH 7.35. Unless otherwise indicated, single-channels recordings were made in the presence of 150-nM [Ca²⁺] and 1.0-mM [Mg²⁺] in the cis compartment. Ryanodine (5 mM) was applied to the cis compartment to confirm identity of all channels. Data were analyzed from digitized current recordings using Fetchan software (Axon Instruments, Union City, Calif.). All data are expressed as mean±SE. The unpaired Student's t-test was used for statistical comparison of mean values between experiments. A value of p<0.05 was considered statistically significant.

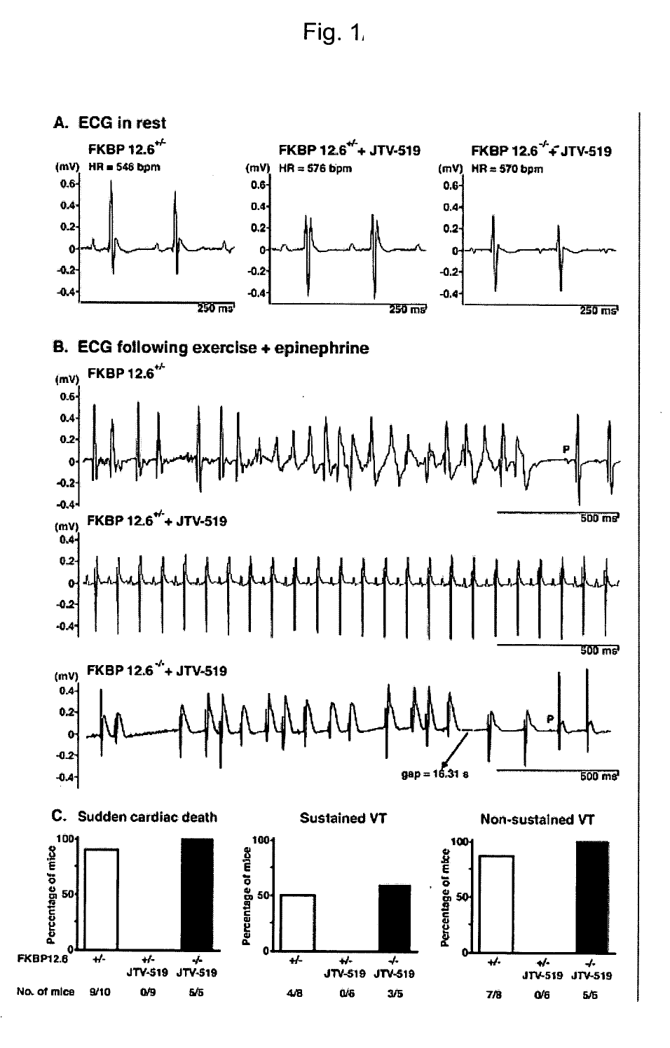

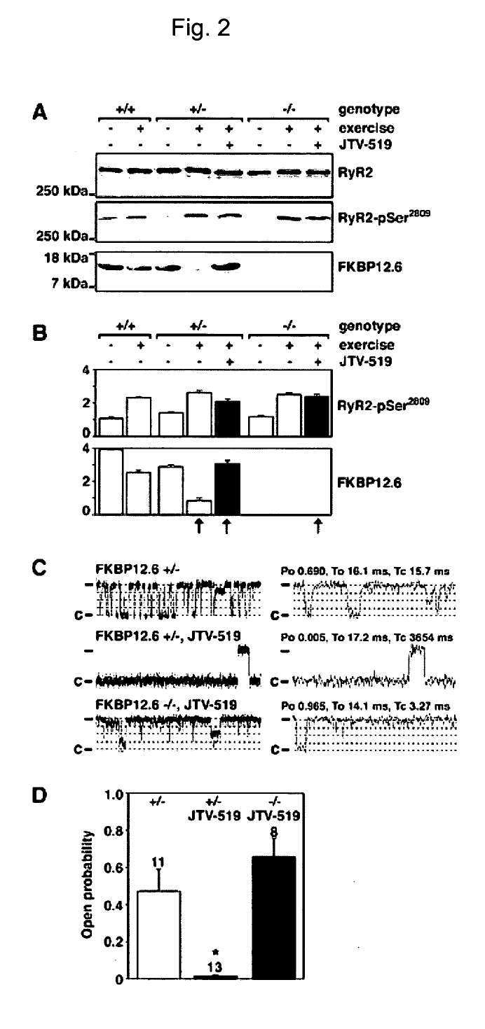

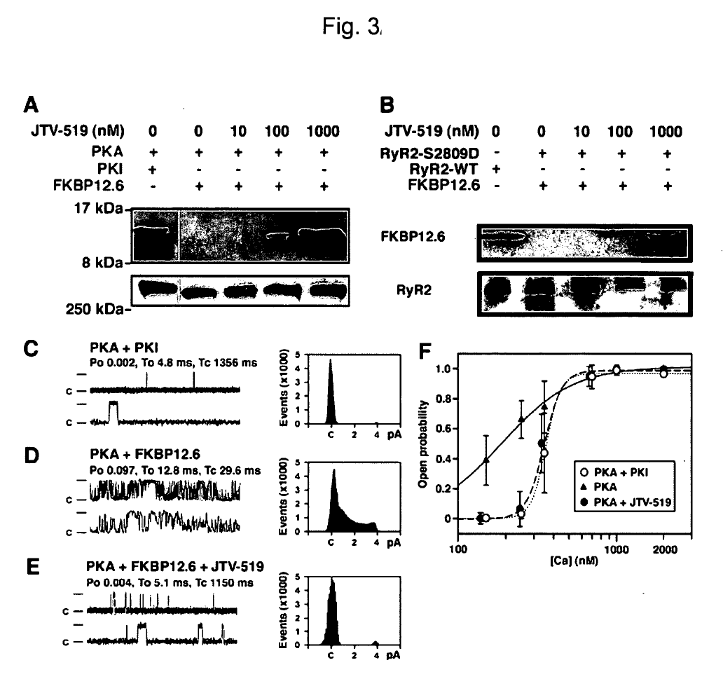

The effects of JTV-519 on RyR2 channels are set forth in FIGS. 1-3 and Table 1 (below). As demonstrated in FIG. 3, the single-channel studies showed increased open probability of RyR2 following PKA phosphorylation (D), as compared to PKA phosphorylation in the presence of the specific PKA inhibitor, PKI₅₋₂₄ (C). Single-channel function was normalized in PKA-phosphorylated RyR2 incubated with FKBP12.6 in the presence of JTV-519 (E). Amplitude histograms (right) revealed increased activity and subconductance openings in PKA-phosphorylated RyR2, but not following treatment with JTV-519 and FKBP12.6. FIG. 3F shows that incubation of PKA-phosphorylated RyR2 with FKBP12.6, in the presence of JTV-519, shifted the Ca²⁺-dependence of RyR2 activation towards the right, making it similar to the Ca²⁺-dependence of unphosphorylated channels.

TABLE 1
Ambulatory ECG data before, during exercise, and following exercise and
injection with epinephrine.

| | SCL (ms) | HR (bpm) | PR (ms) | QRS (ms) | QT (ms) | QTc (ms) |
|---|---|---|---|---|---|---|
| Baseline | | | | | | |
| FKBP12.6^{+/-} | 104 ± 6 | 586 ± 36 | 32 ± 1.5 | 9.9 ± 0.4 | 30 ± 1.0 | 29 ± 0.6 |
| FKBP12.6^{+/-} + JTV-519 | 99 ± 5 | 608 ± 32 | 33 ± 0.6 | 9.3 ± 0.3 | 32 ± 2.7 | 32 ± 1.9 |
| FKBP12.6^{-/-} + JTV-519 | 116 ± 9 | 527 ± 43 | 33 ± 0.4 | 10.0 ± 0.3 | 33 ± 1.3 | 30 + 1.1 |
| Maximum exercise | | | | | | |
| FKBP12.6^{+/-} | 80 ± 2 | 752 ± 18 | 28 ± 0.7 | 8.7 ± 0.4 | 30 ± 1.7 | 33 ± 1.6 |
| FKBP12.6^{+/-} + JTV-519 | 90 ± 7 | 676 ± 49 | 29 ± 1.8 | 9.6 ± 0.4 | 34 ± 2.0 | 36 ± 0.9 |
| FKBP12.6^{-/-} + JTV-519 | 83 ± 3 | 729 ± 22 | 29 ± 2 | 9.3 ± 0.3 | 30 ± 1.2 | 33 ± 0.9 |
| Post-exercise epinephrine | | | | | | |
| FKBP12.6^{+/-} | 94 ± 4 | 645 ± 28 | 35 ± 2.6 | 9.3 ± 0.4 | 33 ± 1.8 | 34 ± 1.9 |
| FKBP12.6^{+/-} + JTV-519 | 102 ± 4 | 592 ± 21 | 37 ± 2.6 | 9.9 ± 0.6 | 32 ± 2.3 | 32 ± 1.7 |
| FKBP12.6^{-/-} + JTV-519 | 103 ± 4 | 585 ± 20 | 35 ± 3.8 | 11.1 ± 0.5 | 36 ± 1.2 | 36 ± 1.3 |

Summary of ambulatory ECG data in FKBP12.6^{+/-} mice treated with JTV-519 (n = 8) or control (n = 6), and FKBP12.6^{-/-} mice treated with JTV-519 (n = 5).
SCL = sinus cycle length;
HR = heart rate;
ms = millisecond;
bpm = beats per minute;
FKBP12.6^{+/-} = FKBP12.6 heterozygous mice;
FKBP12.6^{-/-} = FKBP12.6 deficient mice

### Example 7

### Synthesis of 1,4-Benzothiazepine Intermediate and JTV-519

For the in vivo experiments, the inventors required a gram quantity of JTV-519. However, initial attempts to prepare this compound via the reported 1,4-benzothiazepine intermediate, 7-methoxy-2,3,4,5-tetrahydro-1,4-benzothiazepine (compound 6 in Scheme 1, below), were unsuccessful. The thio group of this intermediate is easily oxidized by air to a disulfide compound, which makes the synthesis of cyclized product (5) impossible. To overcome this problem, the inventors developed a novel process that starts with the readily-available and inexpensive 2-nitro-5-methoxybenzoic acid (1). This process is depicted in Scheme 1 below.

Reduction of the nitro group of compound (1), using H₂ with Pd/C as a catalyst, gave 2-amino-5-methoxybenzoic acid (2) in quantitative yield. Compound (2) was diazotized with NaNO₂, and then treated with Na₂S₂ to provide the stable disulfide compound (3) with 80% yield. Without further purification, the stable disulfide (3) was treated with SOCl₂, and then reacted with 2-chloroethylamine, in the presence of Et₃N, to give an amide (4) in 90% yield. Compound (4) was converted to cyclized compound (5) via a one-pot procedure by reflux with trimethylphosphine and Et₃N in THF. The cyclized amide (5) was then reduced with LiAlH₄ to yield 7-methoxy-2,3,4,5-tetrahydro-1,4-benzothiazepine (6).

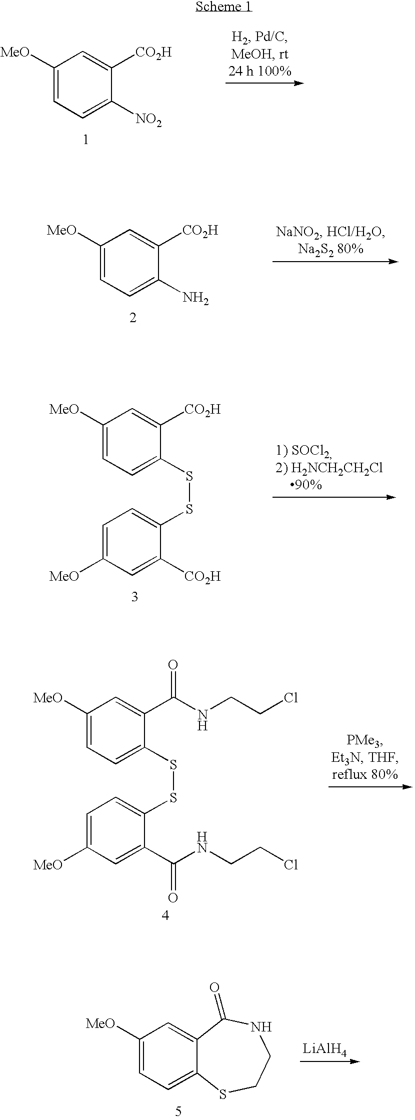

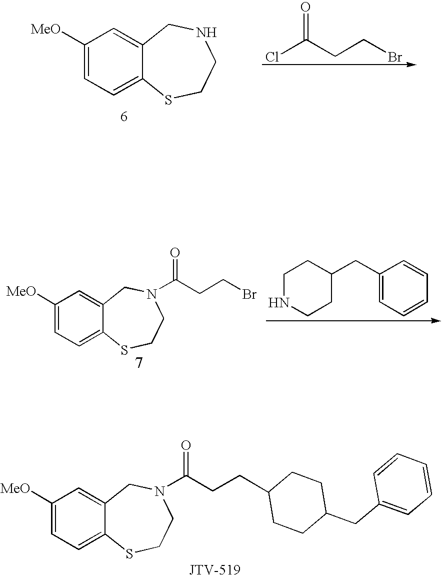

JTV-519 was prepared by reacting compound (6) with 3-bromopropionic chloride, and then reacting the resulting product with 4-benzyl piperidine. The structure of JTV-519 was established by ¹H NMR.

### Example 8

### Synthesis of Radio-Labeled JTV-519

The inventors' novel process for synthesizing radio-labeled JTV-519 is depicted in Scheme 2 below. To prepare radio-labeled JTV-519, JTV-519 was demethylated at the phenyl ring using BBr₃, to give phenol compound (21). The phenol compound (21) was re-methylated with a radio-labeled methylating agent (3H-dimethyl sulfate) in the presence of a base (NaH) to provide ³H-labeled JTV-519 (Scheme 2).

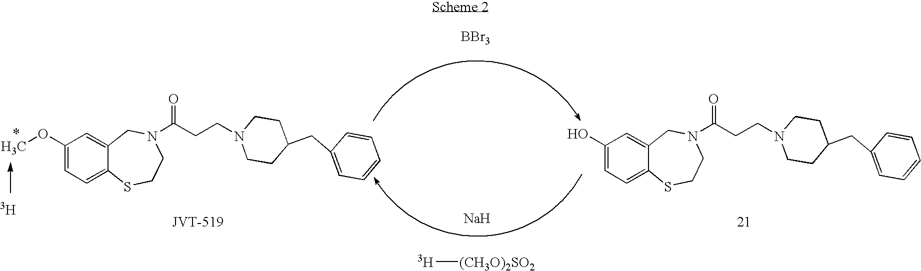

While the foregoing invention has been described in some detail for purposes of clarity and understanding, it will be appreciated by one skilled in the art, from a reading of the disclosure, that various changes in form and detail can be made without departing from the true scope of the invention in the appended claims.

What is claimed is:

1. A method for limiting or preventing a decrease in the level of RyR2-bound FKBP12.6 in a subject who is a candidate for exercise-induced cardiac arrhythmia, comprising administering to the subject an amount of JTV-519 effective to limit or prevent a decrease in the level of RyR2-bound FKBP12.6 in the subject.

2. The method of claim 1, wherein the decrease in the level of RyR2-bound FKBP12.6 is limited or prevented in the subject by decreasing the level of phosphorylated RyR2 in the subject.

3. The method of claim 1, wherein the subject is a human.

4. The method of claim 1, wherein the subject has catecholaminergic polymorphic ventricular tachycardia (CPVT).

5. The method of claim 1, wherein the amount of JTV-519 effective to limit or prevent a decrease in the level of RyR2-bound FKBP12.6 in the subject is an amount of JTV-519 effective to treat or prevent exercise-induced cardiac arrhythmia in the subject.

6. The method of claim 5, wherein the JTV-519 treats or prevents exercise-induced cardiac arrhythmia in the subject.

7. The method of claim 1, wherein the amount of JTV-519 effective to limit or prevent a decrease in the level of RyR2-bound FKBP12.6 in the subject is an amount of JTV-519 effective to prevent exercise-induced sudden cardiac death in the subject.

8. The method of claim 7, wherein the JTV-519 prevents exercise-induced sudden cardiac death in the subject.

9. The method of claim 1, wherein the amount of JTV-519 effective to limit or prevent a decrease in the level of RyR2-bound FKBP12.6 in the subject is from about 5 mg/kg/day to about 20 mg/kg/day.

10. Use of JTV-519 in a method for limiting or preventing a decrease in the level of RyR2-bound FKBP12.6 in a subject who is a candidate for exercise-induced cardiac arrhythmia.

11. A method for treating or preventing exercise-induced cardiac arrhythmia in a subject, comprising administering JTV-519 to the subject in an amount effective to treat or prevent the exercise-induced cardiac arrhythmia in the subject.

12. The method of claim 11, wherein the cardiac arrhythmia is associated with catecholaminergic polymorphic ventricular tachycardia (CPVT).

13. The method of claim 11, wherein the subject is a candidate for exercise-induced sudden cardiac death.

14. The method of claim 11, wherein the amount of JTV-519 effective to treat or prevent the exercise-induced cardiac arrhythmia in the subject is from about 5 mg/kg/day to about 20 mg/kg/day.

15. Use of JTV-519 in a method for treating or preventing exercise-induced cardiac arrhythmia in a subject.

16. A method for preventing exercise-induced sudden cardiac death in a subject, comprising administering to the subject JTV-519 in an amount effective to prevent exercise-induced sudden cardiac death in the subject.

17. The method of claim 16, wherein the exercise-induced sudden cardiac death is associated with catecholaminergic polymorphic ventricular tachycardia (CPVT).

18. The method of claim 16, wherein the amount of JTV-519 effective to prevent the exercise-induced sudden cardiac death in the subject is from about 5 mg/kg/day to about 20 mg/kg/day.

19. A method for identifying an agent for use in preventing exercise-induced sudden cardiac death, comprising the steps of:
(a) obtaining or generating a culture of cells containing RyR2;
(b) contacting the cells with a candidate agent;
(c) exposing the cells to one or more conditions known to increase phosphorylation of RyR2 in cells; and
(d) determining if the agent limits or prevents a decrease in the level of RyR2-bound FKBP12.6 in the cells.

20. The method of claim 19, further comprising the step of:
(e) determining if the agent has an effect on an RyR2-associated biological event in the cells.

21. An agent identified by the method of claim 19.

22. A method for preventing exercise-induced sudden cardiac death in a subject, comprising administering to the subject the agent of claim 21, in an amount effective to prevent exercise-induced sudden cardiac death in the subject.

23. A method for identifying an agent for use in preventing exercise-induced sudden cardiac death, comprising the steps of:
(a) obtaining or generating an animal containing RyR2;
(b) administering a candidate agent to the animal;
(c) exposing the animal to one or more conditions known to increase phosphorylation of RyR2 in cells; and
(d) determining if the agent limits or prevents a decrease in the level of RyR2-bound FKBP12.6 in the animal.

24. The method of claim 23, further comprising the step of:
(e) determining if the agent has an effect on an RyR2-associated biological event in the animal.

25. An agent identified by the method of claim 23.

26. A method for the synthesis of a compound having formula: 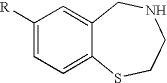
wherein R=OR′, SR′, NR′, alkyl, or halide and R′=alkyl, aryl, or H, and wherein R can be at position 2, 3, 4, or 5, said method comprising the steps of:
(a) treating a compound having formula: 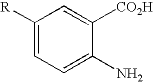
wherein R is as defined above, with a diazotizing agent and a disulfide, to form a compound having formula: 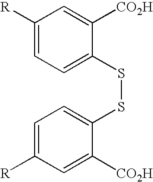
wherein R is as defined above;
(b) treating the compound formed in step (a) with a chloride and a chloroethylamine, to form a compound having formula: 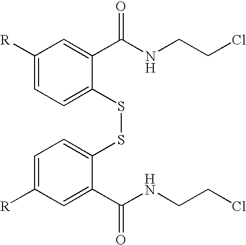
wherein R is as defined above;
(c) treating the compound formed in step (b) with a reducing agent and a base, in the presence of tetrahydrolate, to form a compound having formula: 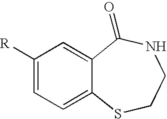
wherein R is as defined above;
(d) treating the compound formed in step (c) with a reducing agent, to form a compound having formula: 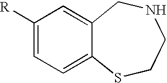
wherein R is as defined above.

27. The method of claim 26, wherein the diazotizing agent in step (a) is NaNO₂.

28. The method of claim 26, wherein the disulfide in step (a) is Na₂S₂.

29. The method of claim 26, wherein the chloride in step (b) is SOCl₂.

30. The method of claim 26, wherein the reducing agent in step (c) is trimethylphosphine (PMe₃).

31. The method of claim 26, wherein the base in step (c) is triethyl amine.

32. The method of claim 26, wherein the reducing agent in step (d) is LiAlH₄.

33. The method of claim 26, wherein the compound in step (a) having formula: 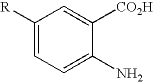
wherein R=OR′, SR′, NR′, alkyl, or halide and R′=alkyl, aryl, or H, and wherein R can be at position 2, 3, 4, or 5, is synthesized by a method comprising the step of:
(e) treating a compound having formula: 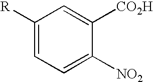
wherein R is as defined above, with a reducing agent, in the presence of an optional catalyst, to form a compound having formula: 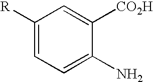
wherein R is as defined above.

34. The method of claim 33, wherein the reducing agent in step (e) is H₂.

35. A method for the synthesis of a compound of having formula: 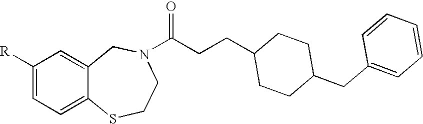
wherein R=OR′, SR′, NR′, alkyl, or halide and R′=alkyl, aryl, or H, and wherein R can be at position 2, 3, 4, or 5, said method comprising the steps of:
(a) treating a compound having formula: 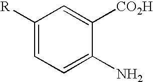
wherein R is as defined above, with a diazotizing agent and a disulfide, to form a compound having formula: 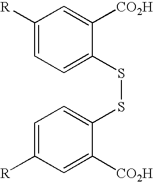
wherein R is as defined above;
(b) treating the compound formed in step (a) with a chloride and a chloroethylamine, to form a compound having formula: 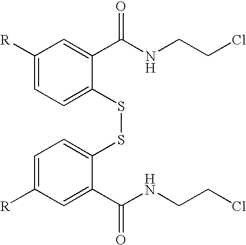
wherein R is as defined above;
(c) treating the compound formed in step (b) with a reducing agent and a base, in the presence of tetrahydrolate, to form a compound having formula: 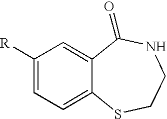
wherein R is as defined above;
(d) treating the compound formed in step (c) with a reducing agent, to form a compound having formula: 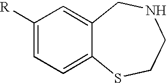
wherein R is as defined above;
(e) treating the compound formed in step (d) with 3-bromopropionic chloride and a compound having formula: 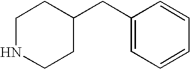
to form a compound having formula: 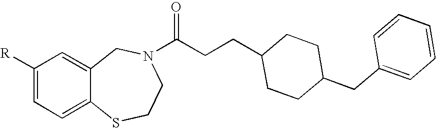
wherein R is as defined above.

36. The method of claim 35, wherein the compound in step (a) having formula: 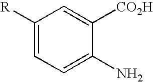
wherein R=OR′, SR′, NR′, alkyl, or halide and R′=alkyl, aryl, or H, and wherein R can be at position 2, 3, 4, or 5, is synthesized by a method comprising the step of:
(f) treating a compound having formula: 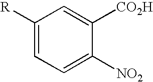
wherein R is as defined above, with a reducing agent, in the presence of an optional catalyst, to form a compound having formula: 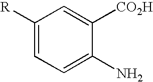
wherein R is as defined above.

37. A method for the synthesis of a compound having formula: 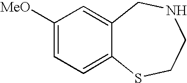
said method comprising the steps of:
(a) treating a compound having formula: 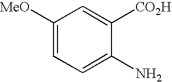
with a diazotizing agent and a disulfide, to form a compound having formula: 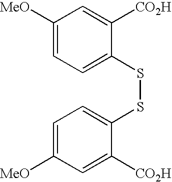
(b) treating the compound formed in step (a) with a chloride and a chloroethylamine, to form a compound having formula: 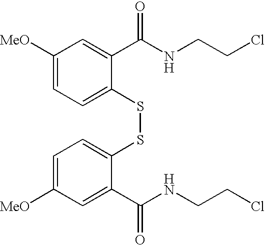
(c) treating the compound formed in step (b) with a reducing agent and a base, in the presence of tetrahydrolate, to form a compound having formula: 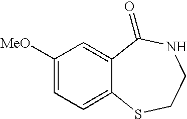
(d) treating the compound formed in step (c) with a reducing agent, to form a compound having formula:

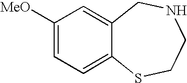

38. The method of claim 37, wherein the compound in step (a) having formula: 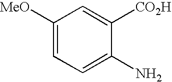
is synthesized by a method comprising the step of:
(e) treating a compound having formula: 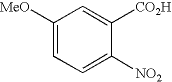
with a reducing agent, in the presence of an optional catalyst, to form a compound having formula:

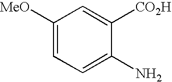

39. A method for the synthesis of a compound having formula: 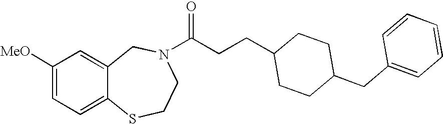
said method comprising the steps of:
(a) treating a compound having formula: 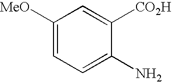
with a diazotizing agent and a disulfide, to form a compound having formula: 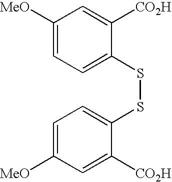
(b) treating the compound formed in step (a) with a chloride and a chloroethylamine, to form a compound having formula: 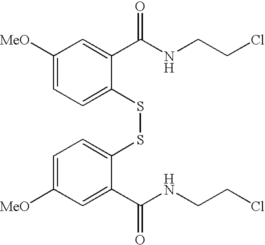
(c) treating the compound formed in step (b) with a reducing agent and a base, in the presence of tetrahydrolate, to form a compound having formula: 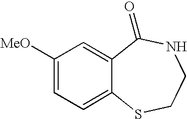
(d) treating the compound formed in step (c) with a reducing agent, to form a compound having formula: 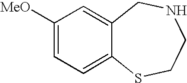
(e) treating the compound formed in step (d) with 3-bromopropionic chloride and a compound having formula: 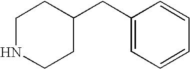
to form a compound having formula:

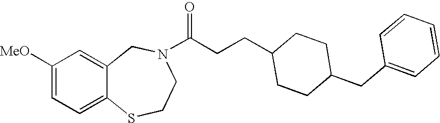

40. The method of claim 39, wherein the compound in step (a) having formula: 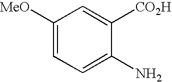
is synthesized by a method comprising the step of:
(f) treating a compound having formula: 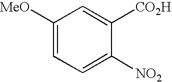
with a reducing agent, in the presence of an optional catalyst, to form a compound having formula: [chemical structure]
